(19) United States
(12) Patent Application Publication
(10) Pub. No.: US 2010/0261241 A1
(43) Pub. Date: Oct. 14, 2010

(54) METHODS FOR THE PRODUCTION OF N-BUTANOL

(76) Inventors: Nikolai Khramtsov, Branford, CT (US); Alexander Amerik, Norwalk, CT (US); Bruce E. Taillon, Middletown, CT (US); Steven Henck, Woodbridge, CT (US)

Correspondence Address: MINTZ LEVIN COHN FERRIS GLOVSKY & POPEO, ONE FINANCIAL CENTER, BOSTON, MA, 02111, US

(21) Appl. No.: 12/739,004

(22) PCT Filed: Oct. 27, 2008
(86) PCT No.: PCT/US08/12186

§ 371 Date: Apr. 21, 2010

Related U.S. Application Data
(60) Provisional application No. 61/000,458, filed on Oct. 26, 2007.

(51) Int. Cl. C12P 7/16; C12N 1/00; C12N 1/21; C12N 1/19; C12N 1/15

(52) U.S. Cl. 435/160; 435/243; 435/252.3; 435/252.31; 435/252.32; 435/252.33; 435/252.34; 435/254.21; 435/254.22; 435/254.23; 435/254.2

(57) ABSTRACT

Embodiments of the present invention include methods for the production of four carbon alcohols, specifically n-butanol, by a consolidated bioprocessing approach for the conversion of cellulosic material to the desired end product. According to some embodiments, recombinant microbial host cells are provided, preferably S. cerevisiae, that are capable of converting cellulosic material to butanol and include butanol biosynthetic pathway genes and cellulase genes.

### CROSS-REFERENCE TO RELATED APPLICATIONS

This application claims priority to U.S. Provisional Patent Application No. 61/000,458, filed Oct. 26, 2007, which is incorporated by reference into this disclosure in its entirety.

### FIELD OF THE INVENTION

This invention relates to methods for the production of four carbon alcohols, specifically n-butanol, by a consolidated bioprocessing approach for the conversion of cellulosic material to the desired end product.

### BACKGROUND OF THE INVENTION

Biofuels are critical to securing energy infrastructures within the United States and around the world by providing alternative fuels, which will not only limit dependence on fossil fuels, but will also reduce the detrimental carbon emissions generated and released into the atmosphere. Current efforts towards the implementation of biofuels have centered on ethanol production and its use.

In addition to ethanol, many anaerobic microorganisms produce other high-energy compounds, including butanol, long-chain alcohols, and ketones, that could either be used as fuels or as substrates for the manufacture of fuels. Butanol in particular offers a number of advantages as a transportation fuel. Butanol is a four-carbon alcohol, a clear neutral liquid miscible with most solvents (alcohols, ether, aldehydes, ketones and hydrocarbons) and is sparingly soluble in water (water solubility 6.3% as compared to ethanol which is totally miscible). It has an octane rating comparable to gasoline, making it a valuable fuel for any internal combustion engine made for burning gasoline. Fuel testing also has proven that butanol does not phase separate in the presence of water, and has no negative impact on elastomer swelling. Because it is less hygroscopic, butanol can be shipped through the existing common-carrier pipelines and stored under humid conditions, unlike ethanol. Butanol not only has a higher energy content that is closer to that of gasoline than ethanol, so it is less of a compromise on fuel economy, but it also can be easily added to conventional gasoline due to its low vapor pressure.

Butanol biosynthesis can be achieved through the acetone, butanol, and ethanol fermentation pathway (the “ABE pathway”). The products of this butanol fermentative production pathway using a solvent-producing species of the bacterium Clostridium acetobutylicum are six parts butanol, three parts acetone, and one part ethanol. Unfortunately, the production of butanol is self-limiting because the products of this fermentation are toxic to cells at a concentration of approximately 13 g butanol/L, which inhibits cell growth resulting in termination of the fermentation process.

Another problem associated with current methods for the production of biofuels is the use of food crops, such as corn and sugar, as the starting material. For example, the use of cereal grains, such as corn, for the production of ethanol competes directly with the food supply, and thus has the unintended consequence of driving up the cost of source material.

An alternative to the use of food crops is biomass, specifically lignocellulosic biomass. Lignocellulosic biomass is more abundant and would be much less expensive to use than food stuffs. Unfortunately, the production of biofuels from cellulose and lignocellulose with current technologies is very difficult because of the complex molecular structure of lignocellulose. Current methods require multiple steps utilizing acid treatment and neutralization, and subsequent treatment with exogenously produced enzymes to hydrolyze the cellulose to sugars.

Cellulose is a very stable polymer with a half-life about 5-8 million years for β-glucosidic bond cleavage at 25° C. (Wolfenden and Snider, 2001). The enzyme-driven cellulose biodegradation process is much faster, and is vital for returning carbon in sediments to the atmosphere (Zhang et al., 2006). The widely accepted mechanism for enzymatic cellulose hydrolysis involves synergistic actions of three different cellulases: endoglucanase, exoglucanase or cellobiohydrolase and β-glucosidase (Lynd et al., 2002). Endoglucanases (1,4-β-D-glucan 4-glucanohydrolases; EC 3.2.1.4) cleave intramolecular β-1,4-glucosidic linkages randomly. Exoglucanases (1,4-β-D-glucan cellobiohydrolases; EC 3.2.1.91) cleave the accessible ends of cellulose molecules to liberate cellobiose. β-glucosidases (β-glucoside glucohydrolases; EC 3.2.1.21) hydrolyze soluble cellobiose and other cellodextrins with a degree of polymerization up to 6 to produce glucose in the aqueous phase. The hydrolysis rates decrease markedly as the degree of substrate polymerization increases (Zhang and Lynd, 2004). Currently, most commercial cellulases are produced using Trichderma and Aspergillus species. The cellulose market is expected to expand dramatically when cellulases are used to hydrolyze pretreated cellulosic materials to sugars, which can be fermented to biofuels on a large scale. Genes encoding cellulases have been cloned from various bacteria, filamentous fungi and plants (Lynd et al., 2002). Several groups have expressed multiple cellulase enzymes in attempts to recreate a fully cellulolytic, fermentative system in Saccharomyces cerevisiae (van Zyl et al., 2007). Since S. cerevisiae lacks the enzymes that hydrolyze cellulose, three types of cellulases were codisplayed on the surface of the yeast cell wall. A yeast strain codisplaying endoglucanase II and cellobiohydrolase II from T. reesei, and A. aculeatus beta-glucosidase I was able to directly produce ethanol from amorphous cellulose with a yield of approximately 2.9 gram per liter (Fujita et al., 2004). Others have expressed two cellulase-encoding genes, endoglucanase of T. reesei and beta-glucosidase of Saccharomycopsis fibuligera, in combination in S. cerevisiae (Den Haan et al., 2007). The highest ethanol titer achieved was ˜1 gram per liter.

Accordingly, there is a need for new methods of producing butanol that eliminates the problems associated with the use of food crops as a starting material and increase the efficiency of production.

### SUMMARY OF THE INVENTION

Methods are provided for producing butanol using a recombinant microorganism having an engineered pathway for the direct conversion of cellulosic material to n-butanol. These methods integrate hydrolysis and fermentation into a single microorganism or a stable mixed culture of microorganisms to increase efficiency of production. More specifically, embodiments of the present invention integrate two or more of the following process steps:
1) Lignin removal from lignocellulose to release cellulose and hemicellulose;
2) De-polymerization of cellulose and hemicellulose to soluble sugars;
3) Fermentation of a mixed-sugar hydrolysate containing six-carbon (hexose) and five-carbon (pentose) sugars;
4) Production of butanol through the solventogenesis pathway; and
5) Shutting down the ethanol and other competing product pathways.

In another aspect, a recombinant microbial host cell is provided, preferably S. cerevisiae, comprising at least one DNA molecule encoding a polypeptide that catalyzes a substrate to product conversion selected from the group consisting of:

(a) pyruvate to acetyl-CoA

(b) acetyl-CoA to acetoacetyl-CoA

(c) acetoacetyl-CoA to (S)-3-hydroxbutanoyl-CoA

(d) (S)-3-hydroxbutanoyl-CoA to crotonoyl-CoA

(e) crotonoyl-CoA to butyryl-CoA

(f) butyryl-CoA to butanal

(g) butanal to butanol

wherein at least one DNA molecule is heterologous to said microbial host cell and wherein said microbial host cell produces butanol.

In yet another aspect, a recombinant microbial host cell is provided, preferably S. cerevisiae, that is capable of converting cellulose to butanol comprising: (1) a DNA molecule encoding at least one cellulase enzyme; and (2) at least one DNA molecule encoding a polypeptide that catalyzes a conversion selected from the group consisting of:

(a) pyruvate to acetyl-CoA

(b) acetyl-CoA to acetoacetyl-CoA

(c) acetoacetyl-CoA to (S)-3-hydroxbutanoyl-CoA

(d) (S)-3-hydroxbutanoyl-CoA to crotonoyl-CoA

(e) crotonoyl-CoA to butyryl-CoA

(f) butyryl-CoA to butanal

(g) butanal to butanol.

In a preferred embodiment, the cellulase enzyme is selected from the group consisting of: endoglucanase II, cellobiohydrolase II, and β-glucosidase I.

In another aspect, a recombinant microbial host cell is provided, preferably S. cerevisiae, that is capable of converting lignocellulose to butanol comprising: (1) a DNA molecule encoding at least one laccase polypeptide; (2) a DNA molecule encoding at least one cellulase polypeptide; and (3) at least one DNA molecule encoding a polypeptide that catalyzes a conversion selected from the group consisting of:

(a) pyruvate to acetyl-CoA

(b) acetyl-CoA to acetoacetyl-CoA

(c) acetoacetyl-CoA to (S)-3-hydroxbutanoyl-CoA

(d) (S)-3-hydroxbutanoyl-CoA to crotonoyl-CoA

(e) crotonoyl-CoA to butyryl-CoA

(f) butyryl-CoA to butanal

(g) butanal to butanol.

In a preferred embodiment, the laccase gene is PDXA1b.

In another aspect, a recombinant microbial host cell is provided, preferably S. cerevisiae, that is capable of converting lignocellulose to butanol comprising: (1) a DNA molecule encoding at least one polypeptide involved in the fermentation of a pentose sugar, preferably xylose; (2) a DNA molecule encoding at least one cellulase polypeptide; and (3) at least one DNA molecule encoding a polypeptide that catalyzes a conversion selected from the group consisting of:

(a) pyruvate to acetyl-CoA

(b) acetyl-CoA to acetoacetyl-CoA

(c) acetoacetyl-CoA to (S)-3-hydroxbutanoyl-CoA

(d) (S)-3-hydroxbutanoyl-CoA to crotonoyl-CoA

(e) crotonoyl-CoA to butyryl-CoA

(f) butyryl-CoA to butanal

(g) butanal to butanol.

It is contemplated that whenever appropriate, any embodiment of the present invention can be combined with one or more other embodiments of the present invention, even though the embodiments are described under different aspects of the present invention.

### BRIEF DESCRIPTION OF THE FIGURES AND SEQUENCE DESCRIPTIONS

The invention can be more fully understood from the following detailed description, figure, and the accompanying sequence descriptions, which form a part of this application.

FIG. 1 shows the Clostridium acetobutylicum butanol biosynthetic pathway starting from acetyl-CoA with the relevant enzymatic activities indicated.

FIG. 2 depicts the AF104 DNA indicating the C. acetobutylicum genes involved in butanol biosynthesis and the unique restriction sites.

FIG. 3 shows a map of plasmid pUG27 carrying the loxP-his5-loxP disruption module and gene disruption using the loxP-his5-loxP disruption cassette. For gene disruption experiments, two oligonucleotides were synthesized (Table 2) with their 3′ ends complementary to sequences left and right of the loxP-his5-loxP module on plasmid pUG27 and with their 5′ ends complementary to the 5′ and 3′ flanking regions of the gene to be disrupted, e.g., ADH1. Plasmid pUG27 was used as PCR template to generate the disruption cassette.

FIG. 4 shows his5 marker rescue by expression of the Cre recombinase. The haploid his⁺ yeast strain with the relevant genotype was transformed with plasmid pSH47. Transformants were grown on glucose plates and then shifted to galactose medium to induce expression of the Cre recombinase. The Cre-induced recombination process between the two loxP sites removes the marker gene.

FIG. 5 shows a calibration curve for quantification of butanol concentration using gas chromatography. Linear calibration curves were developed for ethanol and butanol with ranges of 1000 ppm to 0.8 ppm and 100 ppm to 0.8 ppm, respectively.

FIG. 6 shows ethanol production from PASC (top) and treated paper (bottom) as the source of carbon, respectively, as a function of time. Yeast strains are Y1.C8 with three cell wall attached cellulases; three independent fermentations were performed with this strain. Y1.B9, Y1.C1 and Y1.C2 contain 3 secreted cellulases; Y1.C9 is a control strain containing the same vectors without cellulases.

FIG. 7 shows butanol fermentation during 96 hours from glucose under anaerobic conditions using GasPak™ EX Anaerobic Generating System. All yeast strains are AFY10 derivatives. The negative controls (without butanol genes) are adh1(3a)vector112, adh1(3a)vector195, and adh1(3a)vector181.

FIG. 8 is a gas chromatograph (GC) of the culture media of yeast cells expressing the butanol pathway genes. The n-propanol spike is used to calibrate GC.

FIG. 9 shows butanol production from cellulose (40% PASC) following 336 hours of fermentation. The yeast strains are AFY10 derivatives, where Y1.F9 contains secreted cellulases CBHI and BGLI, and butanol genes; Y1.G4 contains secreted cellulases BGLI and EGII and butanol genes; Y1.C1 contains only secreted cellulases CBHII, BGLI and EGII; Y1.C8 contains only cell wall attached cellulases CBHII, BGLI and EGII; and Y1.C9 is a control strain containing the same vectors without cellulases.

FIG. 10 shows thiolase (THL) spectrophotometric assays. The activity was determined using acetoacetyl-CoA and CoA as substrates. The decrease in acetoacetyl-CoA concentration was measured at 303 nm. Diamonds indicate cell extracts derived from a strain transformed with the pAF104/112 plasmid DNA. Triangles depict control experiments without cell extracts. Squares represent yeast extracts from cells transformed with vector DNA.

FIG. 11 shows HBD spectrophotometric assays. The activity was measured by monitoring decrease in NADH concentration resulting from β-hydroxybutyryl-CoA formation from acetoacetyl-CoA at 345 nm. Squares indicate cell extracts derived from a strain transformed with the pAF104/112 plasmid DNA. Diamonds represent yeast extracts from cells transformed with vector DNA.

FIG. 12 shows an industrial yeast strain (AFY16) that is resistant to butanol at a concentration up to 2%, while the growth of laboratory strains (AFY1, AFY3) is severely impaired at a butanol concentration of 1%.

### DETAILED DESCRIPTION OF THE INVENTION

Recombinant microorganisms are provided that have an engineered pathway for the direct conversion of cellulosic material to butanol. Methods are also provided that integrate hydrolysis and fermentation into a single microorganism or a stable mixed culture of microorganisms to increase efficiency of production. More specifically, embodiments of the present invention integrate two or more of the following process steps:
1) Lignin removal from lignocellulose to release cellulose and hemicellulose;
2) De-polymerization of cellulose and hemicellulose to soluble sugars;
3) Fermentation of a mixed-sugar hydrolysate containing six-carbon (hexose) and five-carbon (pentose) sugars;
4) Production of butanol through the solventogenesis pathway; and
5) Shutting down the ethanol, acetone and other competing product pathways.

Unless defined otherwise, all technical and scientific terms used herein have the same meaning as commonly understood by one of ordinary skill in the art to which this invention belongs. In the case of conflict, the present specification will control. The following definitions and abbreviations are to be used for the interpretation of the claims and the specification.

The term “butanol biosynthetic pathway” refers to an enzyme pathway to produce butanol.

The terms “pyruvate-ferredoxin oxidoreductase” or “pyruvate formate-lyase” are enzymes used to catalyze the conversion from pyruvate to acetyl-CoA. Pyruvate-ferredoxin oxidoreductase and pyruvate formate-lyase are known by the EC Numbers 1.2.7.1 and 2.3.1.54, respectively. (Enzyme Nomenclature 1992, Academic Press, San Diego). The enzymes are available from a number of sources, including, but not limited to GenBank (GenBank Nos. CAC2229 and CAC0980).

The terms “acetyl-CoA C-acetyltransferase” and “thiolase” are used interchangeably herein to refer to an enzyme that catalyzes the conversion from acetyl-CoA to acetoacetyl-CoA. Thiolase is known by EC Number 2.3.1.9. The enzyme is available from a number of sources, including, but not limited to GenBank (GenBank Nos. CAC2873 or CAP0078).

The term “3-hydroxybutyryl-CoA dehydrogenase” refers to an enzyme that catalyzes the conversion from acetoacetyl-CoA to (S)-3-hydroxybutanoyl-CoA. 3-hydroxybutyryl-CoA dehydrogenase is known by EC Number 1.1.1.157. The enzyme is available from a number of sources, including, but not limited to GenBank (GenBank Nos. CAC2708 or CAC2009).

The terms “3-hydroxybutyryl-CoA dehydratase” or “crotonase” are used interchangeably herein to refer to an enzyme that catalyzes the conversion from (S)-3-hydroxybutanoyl-CoA to crotonoyl-CoA. 3-hydroxybutyryl-CoA dehydratase is known by EC Number 4.2.1.55. The enzyme is available from a number of sources, including, but not limited to GenBank (GenBank Nos. CAC2712, CAC2012, or CAC2016).

The term “butyryl-CoA dehydrogenase” refers to an enzyme that catalyzes the conversion from crotonoyl-CoA to butyryl-CoA. Butyryl-CoA dehydrogenase is known by EC Number 1.3.99.2. The enzyme is available from a number of sources, including, but not limited to GenBank (GenBank No. CAC2711).

The terms “butyraldehyde dehydrogenase”, “aldehyde-alcohol dehydrogenase”, “alcohol dehydrogenase” and “acetaldehyde dehydrogenase” are used interchangeably herein and refer to an enzyme that catalyzes the conversion from butyryl-CoA to butanal. Preferred butyraldehyde dehydrogenases are know by EC Number 1.2.1.57. Other EC Numbers include 1.1.1.1 and 1.2.1.10. The enzyme is available from a number of sources, including, but not limited to GenBank (GenBank Nos. CAP0162 or CAP0035).

The term “butanol dehydrogenase” refers to an enzyme that catalyzes the conversion from butanal to butanol. This enzyme is known by EC Number 1.1.1. The enzyme is available from a number of sources, including, but not limited to GenBank (GenBank Nos. CAP0162, or CAP0035, or CAP0059, or CAC3298, or CAC3299, or CAC3392).

The term “carbon substrate” refers to a carbon source capable of being metabolized by host organisms of the present invention, and particularly carbon sources selected from the group consisting of monosaccharides, oligosaccharides, polysaccharides, or mixtures thereof.

The term “gene” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as naturally found in a host organism with its own regulatory sequences. “Chimeric gene” refers to any gene that is not a native gene, comprising regulatory and coding sequences that are not found together in the host organism. Accordingly, a chimeric gene may comprise regulatory sequences and coding sequences that are derived from different sources, or regulatory sequences and coding sequences derived from the same source, but arranged in a manner different than that found in that source. “Endogenous gene” refers to a native gene in its natural location in the genome of an organism. A “foreign gene” or “heterologous gene” refers to a gene not normally found in the host organism, but that is introduced into the host organism by gene transfer. Foreign genes can comprise native genes inserted into a non-native organism, or chimeric genes. It is also understood, that foreign genes encompass genes whose coding sequence has been modified to enhance its expression in a particular host, for example, codons can be substituted to reflect the preferred codon usage of the host. A “transgene” is a gene that has been introduced into the genome by a transformation procedure.

As used herein the term “coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structures.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from nucleic acid fragments of the invention. Expression may also refer to translation of mRNA into a polypeptide.

As used herein, the term “transformation” refers to the insertion of an exogenous nucleic acid into a cell, irrespective of the method used for the insertion, for example, lipofection, transduction, infection or electroporation. The exogenous nucleic acid can be maintained as a non-integrated vector, for example, a plasmid, or alternatively, can be integrated into the cell's genome. Host organisms containing the transformed nucleic acid fragments are referred to as “transgenic” or “recombinant” or “transformed” organisms.

The terms “plasmid”, “vector” and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector or linear DNA fragment containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host.

Standard molecular biology techniques used herein are well known in the art and are described by Sambrook J, Fritsch E F, Maniatis T. 1989. Molecular Cloning: A Laboratory Manual. Cold Spring Harbor Laboratory Press: Cold Spring Harbor, N.Y. Techniques for manipulation of S. cerevisiae used herein are well known in the art and are described in Sherman F, Fink G R, Hicks J B. 1986. Methods in Yeast Genetics. Cold Spring Harbor Laboratory Press: Cold Spring Harbor, N.Y., and in Guthrie C, Fink GR, (Eds.). 2002. Methods in Enzymology, Volume 351, Guide to Yeast Genetics and Molecular and Cell Biology (Part C), Elsevier Academic Press, San Diego, Calif.

### Consolidated BioProcessing Approach

Consolidated bioprocessing (CBP) is a processing strategy for cellulosic biomass which involves consolidating two or more of the following steps into a single process step:
1) Lignin removal from lignocellulose to release cellulose and hemicellulose;
2) De-polymerization of cellulose and hemicellulose to soluble sugars;
3) Fermentation of a mixed-sugar hydrolysate containing six-carbon (hexose) and five-carbon (pentose) sugars;
4) Production of butanol through the solventogenesis pathway; and
5) Shutting down ethanol, acetone and other competing product pathways.
   1) Lignin Removal from Lignocellulose

Laccases are enzymes that catalyze the oxidation of a variety of phenolic compounds as well as diamines and aromatic amines. In fungi, laccases are involved in the degradation of lignocellulosic materials. Ligninolytic enzymes are notoriously difficult to express in non-fungal systems. However, some embodiments of the present invention use laccase genes to break down lignin and release the cellulose or hemicellulose. Other enzymes suitable for expression in yeast to breakdown lignin include: lignin peroxide and manganese-dependent peroxidase.

### 2) Depolymerization of Cellulose to Soluble Sugars

Enzymatic degradation of cellulose involves the coordinate action of at least three different types of cellulases. Such enzymes are given an Enzyme Commission (EC) designation according to the Nomenclature Committee of the International Union of Biochemistry and Molecular Biology (Eur. J. Biochem. 264: 607 609 and 610 650, 1999). Endo-β-(1,4)-glucanases (EC 3.2.1.4) cleave the cellulose strand randomly along its length, thus generating new chain ends. Exo-β-(1,4)-glucanases (EC 3.2.1.91) are processive enzymes and cleave cellobiosyl units (beta-(1,4)-glucose dimers) from free ends of cellulose strands. Lastly, beta-D-glucosidases (cellobiases: EC 3.2.1.21) hydrolyze cellobiose to glucose. All three of these general activities are required for efficient and complete hydrolysis of a polymer such as cellulose to a subunit, such as the simple sugar, glucose.

Yeast is, of course, a natural sugar fermentor-converting sugar into ethanol. Cellulose degrading yeast strains can be made, for example, by codisplaying cellulolytic enzymes from the filamentous fungus T. reesei on the cell surface of S. cerevisiae. These engineered yeasts then directly produce ethanol from pure cellulose (Fujita et al, 2004; Den Haan et al, 2007).

### 3) Fermentation of a Mixed-Sugar Hydrolysate Containing Six-Carbon (Hexoses) and Five-Carbon (Pentoses) Sugars

One of the most effective ethanol-producing yeasts, S. cerevisiae, has several advantages such as high ethanol production from hexoses and high tolerance to ethanol and other inhibitory compounds in the acid hydrolysates of lignocellulose biomass. However, because standard strains of this yeast cannot utilize pentoses, such as xylose, and celloligosaccharides (two to six glucose units), fermentation from a lignocellulose hydrolysate will not be completely efficient. According to some embodiments of the present invention, a recombinant yeast strain is provided that can ferment xylose and cellooligosaccharides by integrating genes for the intercellular expression of xylose reductase and xylitil dehydrogenase from Pichia stipitis and a gene for displaying β-glucosidase from A. acleatus.

### 4) Production of Butanol Through the Solventogenesis Pathway

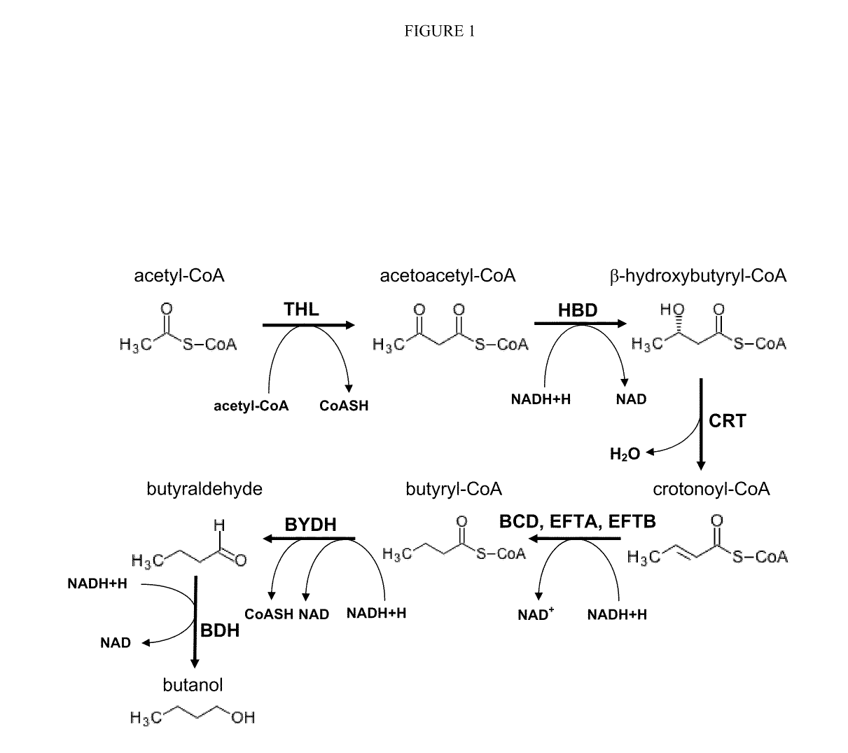

Acetone, butanol and other solvents can be produced to commercially important levels by several Clostridium species. Isolates of C. acetobutylicum, first identified between 1912 and 1914, were used to develop an industrial starch-based acetone, butanol, and ethanol (ABE) fermentation process, to produce acetone for production of explosives by Chaim Weizmann during World War I. During the 1920s and 1930s, increased demand for butanol led to the establishment of large fermentation factories and a more efficient molasses-based process. However, the establishment of more cost-effective petrochemical processes during the 1950s led to the abandonment of the ABE process in all but a few countries. Commercial production facilities were still operating in Russia until the 1980s. The type strain, C. acetobutylicum ATCC 824, was isolated in 1924 from garden soil in Connecticut and is one of the best-studied solventogenic clostridia. This strain is known to utilize a broad range of monosaccharides, disaccharides, starches, and other substrates, such as whey and xylan, but not crystalline cellulose. Genes from the pathway in FIG. 1 are synthesized and transformed into a S. cerevisiae strain that is selected for maximal butanol production.

### 5) Shutting Down Ethanol and Other Competing Product Pathways

Yeast is a natural sugar fermenting cell line converting sugar into ethanol. Several methods known in the art can be used to shut down ethanol and other competing pathways. For example, site directed mutagenesis (SDM) can be used to make genes within the ethanol pathway non-functional by specific, selective mutation. Genes can also be inserted into yeast genome to knock-out genes within the ethanol pathway via homologous recombination.

### Microbial Hosts for Butanol Production

Microbial hosts for butanol production may be selected from bacteria, cyanobacteria, filamentous fungi and yeasts. The microbial hosts selected for the production of butanol are preferably tolerant to butanol and should be able to convert carbohydrates to butanol. Suitable microbial hosts include hosts with one or more, preferably all, of the following characteristics: intrinsic tolerance to butanol, high rate of glucose utilization, availability of genetic tools for gene manipulation, and the ability to generate stable chromosomal alterations.

The ability to genetically modify the host is useful for the production of a recombinant microorganism. The mode of gene transfer technology may be any method known in the art, such as by electroporation, conjugation, transduction or natural transformation. A broad range of host conjugative plasmids and drug resistance markers are available and known to one of skill in the art. The cloning vectors are tailored to the host organism based on the nature of the markers that are used in that host.

The microbial host also can be manipulated in order to inactivate competing pathways for carbon flow by deleting various genes. This generally requires the availability of either transposons to direct inactivation or chromosomal integration vectors. Additionally, the production host should be amenable to chemical mutagenesis so that mutations to improve intrinsic butanol tolerance may be obtained.

Suitable microbial hosts for the production of butanol include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces. Preferred hosts include: Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis, Saccharomyces carlsburgenesis and Saccharomyces cerevisiae. A preferred microbial host is a Saccharomyces species, for example, Saccharomyces carlsburgenesis and Saccharomyces cerevisiae. A particularly preferred microbial host is Saccharomyces cerevisiae.

### Construction of Production Host

Recombinant organisms containing the genes encoding the enzymatic pathway for the conversion of cellulose substrate to butanol are constructed using techniques well known in the art. Genes encoding the enzymes of one of the butanol biosynthetic pathways of the invention, for example acetyl-CoA C-acetyltransferase (thiolase), 3-hydroxybutyryl-CoA dehydrogenase, 3-hydroxybutyryl-CoA dehydratase (crotonase), butyryl-CoA dehydrogenase, butyraldehyde dehydrogenase, and butanol dehydrogenase may be isolated from various sources, as described above.

Methods of obtaining desired genes from a bacterial genome are common and well known in the art of molecular biology. For example, if the sequence of the gene is known, suitable genomic libraries may be created by restriction endonuclease digestion and may be screened with probes complementary to the desired gene sequence. Once the sequence is isolated, the DNA may be amplified using standard primer-directed amplification methods such as polymerase chain reaction (U.S. Pat. No. 4,683,202) to obtain amounts of DNA suitable for transformation using appropriate vectors.

Once the relevant pathway genes are identified and isolated they may be transformed into suitable expression hosts by means well known in the art. Vectors or cassettes useful for the transformation of a variety of host cells are common and commercially available from companies such as EPICENTRE® (Madison, Wis.), Invitrogen Corp. (Carlsbad, Calif.), Stratagene (La Jolla, Calif.), and New England Biolabs, Inc. (Beverly, Mass.). Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. Both control regions may be derived from genes homologous to the transformed host cell, although it is to be understood that such control regions may also be derived from genes that are not native to the specific species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention. Promoters useful for expression in Saccharomyces include, but are not limited to CYC1, HIS3, GAL1, GAL10, ADH1, PGK, PHO5, GAPDH, ADC1, TRP1, URA3, LEU2, ENO, TPI, CUP1, FBA, GPD, and GPM.

Termination control regions may also be derived from various genes native to the preferred hosts. Optionally, a termination site may be unnecessary; however, it is most preferred if included.

All sequence citations, references, patents, patent applications or other documents cited are hereby incorporated by reference.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

### Construction of Expression Plasmids Encoding Cellulase Genes

Expression constructs encoding cellulases for co-display on the yeast cell wall surface were constructed by fusing the cellulase genes with the DNA encoding the secretion signal sequence of glucoamylase from Rhizopus oryzae. The secretion signal is responsible for delivery of the cellulase to the cell wall. The gene, encoding the C-terminal half of S. cerevisiae α-agglutinin was linked to the 3′-end of the cellulase. The α-agglutinin part of the recombinant protein allows for the attachment to the cell wall. Furthermore, all three cellulases were also expressed in secreted soluble forms that are not attached to the cell wall. Expression constructs for secreted forms lacked the α-agglutinin portion.

DNA sequences of cellulase genes are known, and the following genes were used: T. reesei endoglucanase II (GenBank accession number DQ178347); T. reesei cellobiohyrdolase II (GenBank accession number M55080) and A. aculeatus β-glucosidase I (GenBank accession number D64088). The cellulase DNA constructs were commercially synthesized by Blue Heron Bio using their GeneMaker® synthesis platform. Unique restriction endonuclease sites were added to the sequences to facilitate subcloning into expression vectors. Several restriction sites were removed from coding sequences via one nucleotide substitutions that did not change the amino acid sequence.

The cellulase DNA constructs were commercially synthesized by Blue Heron Bio were cloned into the Blue Heron pUC119 vector. The sequences of the vector inserts are shown below:

pUC119-AF101 (cellobiohydrolase II (CBHII)
construct):

(SEQ ID NO: 1)

AAGCTTGCATGCAGTTTATCATTATCAATACTCGCCATTTCAAAGAATAC
GTAAATAATTAATAGTAGTGATTTTCCTAACTTTATTTAGTCAAAAAATT
AGCCTTTTAATTCTGCTGTAACCCGTACATGCCCAAAATAGGGGGCGGGT
TACACAGAATATATAACATCGTAGGTGTCTGGGTGAACAGTTTATTCCTG
GCATCCACTAAATATAATGGAGCCCGCTTTTTAAGCTGGCATCCAGAAAA
AAAAAGAATCCCAGCACCAAAATATTGTTTTCTTCACCAACCATCAGTTC
ATAGGTCCATTCTCTTAGCGCAACTACAGAGAACAGGGGCACAAACAGGC
AAAAAACGGGCACAACCTCAATGGAGTGATGCAACCTGCCTGGAGTAAAT
GATGACACAAGGCAATTGACCCACGCATGTATCTATCTCATTTTCTTACA
CCTTCTATTACCTTCTGCTCTCTCTGATTTGGAAAAAGCTGAAAAAAAAG
GTTGAAACCAGTTCCCTGAAATTATTCCCCTACTTGACTAATAAGTATAT
AAAGACGGTAGGTATTGATTGTAATTCTGTAAATCTATTTCTTAAACTTC
TTAAATTCTACTTTTATAGTTAGTCTTTTTTTTAGTTTTAAAACACCAGA
ACTTAGTTTCGACGGATCTGCAGGTCGACATGCAACTGTTCAATTTGCCA
TTGAAAGTTTCATTCTTTCTCGTCCTCTCTTACTTTTCTTTGCTCGTTTC
TGCTGACTACAAGGACGATGACGACAAATCTAGACAGGCTTGCTCAAGCG
TCTGGGGCCAATGTGGTGGCCAGAATTGGTCGGGTCCGACTTGCTGTGCT
TCCGGAAGCACATGCGTCTACTCCAACGACTATTACTCCCAGTGTCTTCC
CGGCGCTGCAAGCTCAAGCTCGTCCACGCGCGCCGCATCGACGACTTCAC
GAGTATCCCCCACAACATCCCGGTCGAGTTCCGCGACGCCTCCACCTGGT
TCTACTACTACCAGAGTACCTCCAGTCGGATCGGGAACCGCTACGTATTC
AGGCAACCCTTTTGTTGGGGTCACTCCTTGGGCCAATGCATATTACGCCT
CTGAAGTTAGCAGCCTCGCTATTCCTAGCTTGACTGGAGCCATGGCCACT
GCCGCAGCAGCTGTCGCAAAGGTTCCCTCTTTTATGTGGCTAGATACTCT
TGACAAGACCCCTCTCATGGAGCAAACCTTGGCCGACATCCGCACCGCCA
ACAAGAATGGCGGTAACTATGCCGGACAGTTTGTGGTGTATGACTTGCCG
GATCGCGATTGCGCTGCCCTTGCCTCGAATGGCGAATACTCTATTGCCGA
TGGTGGCGTCGCCAAATATAAGAACTATATCGACACCATTCGTCAAATTG
TCGTGGAATATTCCGATATCCGGACCCTCCTGGTTATTGAGCCTGACTCT
CTTGCCAACCTGGTGACCAACCTCGGTACTCCAAAGTGTGCCAATGCTCA
GTCAGCCTACCTTGAGTGCATCAACTACGCCGTCACACAGCTGAACCTTC
CAAATGTTGCGATGTATTTGGACGCTGGCCATGCAGGATGGCTTGGCTGG
CCGGCAAACCAAGACCCGGCCGCTCAGCTATTTGCAAATGTTTACAAGAA
TGCATCGTCTCCGAGAGCACTTCGCGGATTGGCAACCAATGTCGCCAACT
ACAACGGGTGGAACATTACCAGCCCCCCATCGTACACGCAAGGCAACGCT
GTCTACAACGAGAAGCTGTACATCCACGCTATTGGACGTCTTCTTGCCAA
TCACGGCTGGTCCAACGCCTTCTTCATCACTGATCAAGGTCGATCGGGAA
AGCAGCCTACCGGACAGCAACAGTGGGGAGACTGGTGCAATGTGATCGGC
ACCGGATTTGGTATTCGCCCATCCGCAAACACTGGGGACTCGTTGCTGGA
TTCGTTTGTCTGGGTCAAGCCAGGCGGCGAGTGTGACGGCACCAGCGACA
GCAGTGCGCCACGATTTGACTCCCACTGTGCGCTCCCAGATGCCTTGCAA
CCGGCGCCTCAAGCTGGTGCTTGGTTCCAAGCCTACTTTGTGCAGCTTCT
CACAAACGCAAACCCATCGTTCCTGGGATCCAGCGCCAAAAGCTCTTTTA
TCTCAACCACTACTACTGATTTAACAAGTATAAACACTAGTGCGTATTCC
ACTGGTTCCATTTCCACAGTAGAAACAGGCAATCGAACTACATCAGAAGT
GATCAGTCATGTGGTGACTACCAGCACAAAACTGTCTCCAACTGCTACTA
CCAGCCTGACAATTGCACAAACCAGTATCTATTCTACTGACTCAAATATC
ACAGTAGGAACAGATATTCACACCACATCAGAAGTGATTAGTGATGTGGA
AACCATTAGCAGAGAAACAGCTTCGACCGTTGTAGCCGCTCCAACCTCAA
CAACTGGATGGACAGGCGCTATGAATACTTACATCCCGCAATTTACATCC
TCTTCTTTCGCAACAATCAACAGCACACCAATAATCTCTTCATCAGCAGT
ATTTGAAACCTCAGATGCTTCAATTGTCAATGTGCACACTGAAAATATCA
CGAATACTGCTGCTGTTCCATCTGAAGAGCCCACTTTTGTAAATGCCACG
AGAAACTCCTTAAATTCCTTTTGCAGCAGCAAACAGCCATCCAGTCCCTC
ATCTTATACGTCTTCCCCACTCGTATCGTCCCTCTCCGTAAGCAAAACAT
TACTAAGCACCAGTTTTACGCCTTCTGTGCCAACATCTAATACATATATC
AAAACGGAAAATACGGGTTACTTTGAGCACACGGCTTTGACAACATCTTC
AGTTGGCCTTAATTCTTTTAGTGAAACAGCACTCTCATCTCAGGGAACGA
AAATTGACACCTTTTTAGTGTCATCCTTGATCGCATATCCTTCTTCTGCA
TCAGGAAGCCAATTGTCCGGTATCCAACAGAATTTCACATCAACTTCTCT
CATGATTTCAACCTATGAAGGTAAAGCGTCTATATTTTTCTCAGCTGAAC
TCGGTTCGATCATTTTTCTGCTTTTGTCGTACCTGCTATTCTAACCCGGG
TACCTCATGTAATTAGTTATGTCACGCTTACATTCACGCCCTCCCCCCAC
ATCCGCTCTAACCGAAAAGGAAGGAGTTAGACAACCTGAAGTCTAGGTCC
CTATTTATTTTTTTATAGTTATGTTAGTATTAAGAACGTTATTTATATTT
CAAATTTTTCTTTTTTTTCTGTACAGACGCGTGTACGCATGTAACATTAT
ACTGAAAACCTTGCTTGAGAAGGTTTTGGGACGCTCGAAGGCTTTAATTT
GCGGCCGAGCTCGAATTC

Where nucleotides:
1 to 12 are HindIII and SphI restriction sites;
13 to 667 is the GPDH promoter (GenBank accession number DQ019861);
668 to 679 are PstI and SalI restriction sites;
680 to 754 is ATG and secretion signal from the R. oryzae glucoamylase gene (GenBank accession number D00049);
755 to 778 is a FLAG tag;
779 to 784 is a XbaI restriction site;
785 to 2125 is mature cellobiohydrolase II (CBHII) from T. reesi (GenBank accession number M55080), with the following nucleotide changes introduced (numbering according to the M55080 DNA sequence): A75G, G225A, T237A, C267T, T441C, G561C, T957A, and G1345C;
2126 to 2131 is a BamHI restriction site;
2132 to 3094 is the α-agglutinin 3′-gene portion with STOP codon (GenBank accession number AAA34417 or M28164), with the following nucleotide changes introduced (numbering according to the M28164 DNA sequence): T1422A, T1887C, and A2265G;
3095 to 3104 are SmaI-KpnI restriction sites;
3105 to 3356 is the CYC1 terminator (GenBank accession number EF210199); and 3357 to 3368 are SacI-EcoRI restriction sites.

pUC119-AF102 (β-glucosidase I (BGLI) construct):

(SEQ ID NO: 2)

TCTAGAGATGAACTGGCGTTCTCTCCTCCTTTCTACCCCTCTCCGTGGGC
CAATGGCCAGGGAGAGTGGGCGGAAGCCTACCAGCGTGCAGTGGCCATTG
TATCCCAGATGACTCTGGATGAGAAGGTCAACCTGACCACCGGAACTGGA
TGGGAGCTGGAGAAGTGCGTCGGTCAGACTGGTGGTGTCCCAAGACTGAA
CATCGGTGGCATGTGTCTTCAGGACAGTCCCTTGGGTATTCGTGATAGTG
ACTACAATTCGGCTTTCCCTGCTGGTGTCAACGTTGCTGCGACATGGGAC
AAGAACCTTGCTTATCTACGTGGTCAGGCTATGGGTCAAGAGTTCAGTGA
CAAAGGAATTGATGTTCAATTGGGACCGGCCGCGGGTCCCCTCGGCAGGA
GCCCTGATGGAGGTCGCAACTGGGAAGGTTTCTCTCCAGACCCGGCTCTT
ACTGGTGTGCTCTTTGCGGAGACGATTAAGGGTATTCAAGACGCTGGTGT
CGTGGCGACAGCCAAGCATTACATTCTCAATGAGCAAGAGCATTTCCGCC
AGGTCGCAGAGGCTGCGGGCTACGGATTCAATATCTCCGACACGATCAGC
TCTAACGTTGATGACAAGACCATTCATGAAATGTACCTCTGGCCCTTCGC
GGATGCCGTTCGCGCCGGCGTTGGCGCCATCATGTGTTCCTACAACCAGA
TCAACAACAGCTACGGTTGCCAGAACAGTTACACTCTGAACAAACTTCTG
AAGGCCGAACTCGGCTTCCAGGGCTTTGTGATGTCTGACTGGGGTGCTCA
CCACAGTGGTGTTGGCTCTGCTTTGGCCGGCTTGGATATGTCAATGCCTG
GCGATATCACCTTCGATTCTGCCACTAGTTTCTGGGGAACCAACCTGACC
ATTGCTGTGCTCAACGGAACCGTCCCGCAGTGGCGCGTTGACGACATGGC
TGTCCGTATCATGGCTGCCTACTACAAGGTTGGCCGCGACCGCCTGTACC
AGCCGCCTAACTTCAGCTCCTGGACTCGCGATGAATACGGCTTCAAGTAT
TTCTACCCCCAGGAAGGGCCCTATGAGAAGGTCAATCACTTTGTCAATGT
GCAGCGCAACCACAGCGAGGTTATTCGCAAGTTGGGAGCAGACAGTACTG
TTCTACTGAAGAACAACAATGCCCTGCCGCTGACCGGAAAGGAGCGCAAA
GTTGCGATCCTGGGTGAAGATGCTGGTTCCAACTCGTACGGTGCCAATGG
CTGCTCTGACCGTGGCTGTGACAACGGTACTCTTGCTATGGCTTGGGGTA
GCGGCACTGCCGAATTTCCATATCTCGTGACCCCTGAGCAGGCTATTCAA
GCCGAGGTGCTCAAGCATAAGGGCAGCGTCTACGCCATCACGGACAACTG
GGCGCTGAGCCAGGTGGAGACCCTCGCTAAACAAGCCAGTGTCTCTCTTG
TATTTGTCAACTCGGACGCGGGAGAGGGCTATATCTCCGTGGACGGAAAC
GAGGGCGACCGCAACAACCTCACCCTCTGGAAGAACGGCGACAACCTCAT
CAAGGCTGCTGCAAACAACTGCAACAACACCATCGTTGTCATCCACTCCG
TTGGACCTGTTTTGGTTGACGAGTGGTATGACCACCCCAACGTTACTGCC
ATCCTCTGGGCGGGCTTGCCTGGCCAGGAGTCTGGCAACTCCTTGGCTGA
CGTGCTCTACGGCCGCGTCAACCCAGGCGCCAAATCTCCATTCACCTGGG
GCAAGACGAGGGAGGCGTACGGGGATTACCTTGTCCGTGAACTCAACAAC
GGCAACGGAGCACCCCAAGATGATTTCTCGGAAGGTGTTTTCATTGACTA
CCGCGGATTCGACAAGCGCAATGAGACCCCGATCTACGAGTTCGGACATG
GTCTGAGCTACACCACTTTCAACTACTCTGGCCTTCACATCCAGGTTCTC
AACGCTTCCTCCAACGCTCAAGTAGCCACTGAGACTGGCGCCGCTCCCAC
CTTCGGACAAGTCGGCAATGCCTCTGACTACGTGTACCCTGAGGGATTGA
CCAGAATCAGCAAGTTCATCTATCCCTGGCTTAATTCCACAGACCTGAAG
GCCTCATCTGGCGACCCGTACTATGGAGTCGACACCGCGGAGCACGTGCC
CGAGGGTGCTACTGATGGCTCTCCGCAGCCCGTTCTGCCTGCCGGTGGTG
GCTCTGGTGGTAACCCGCGCCTCTACGATGAGTTGATCCGTGTTTCGGTG
ACAGTCAAGAACACTGGTCGTGTTGCCGGTGATGCTGTGCCTCAATTGTA
TGTTTCCCTTGGTGGACCCAATGAGCCCAAGGTTGTGTTGCGCAAATTCG
ACCGCCTCACCCTCAAGCCCTCCGAGGAGACGGTGTGGACGACTACCCTG
ACCCGCCGCGATCTGTCTAACTGGGACGTTGCGGCTCAGGACTGGGTCAT
CACTTCTTACCCGAAGAAGGTCCATGTTGGTAGCTCTTCGCGTCAGCTGC
CCCTTCACGCGGCGCTCCCGAAGGTGCAAGGATCCTAAGGTACC

Where nucleotides:
1 to 6 is a XbaI restriction site;
7 to 2529 is mature β-glucosidase I from A. aculeatus (GenBank accession numbers D64088 or BAA10968), with the following nucleotide changes introduced (numbering according to the D64088 DNA sequence): A398T, G905A, G920A, T1049A, and T1079A; A1388T; C1478T; G1886A, G1952A, T1973A;
2530 to 2535 is a BamHI restriction site;
2536 to 2538 is a TAA STOP codon; and
2539 to 2544 is a KpnI restriction site.

pUC119-AF103 (endoglucanase (EGII) construct):

(SEQ ID NO: 3)

TCTAGACAGCAGACTGTCTGGGGCCAGTGTGGAGGTATTGGTTGGAGCGG
ACCTACGAATTGTGCTCCTGGCTCAGCTTGTTCGACCCTCAATCCTTATT
ATGCGCAATGTATTCCGGGAGCCACTACTATCACCACTTCGACCCGGCCA
CCATCCGGTCCAACCACCACCACCAGGGCTACCTCAACAAGCTCATCAAC
TCCACCCACTAGCTCTGGGGTCCGATTTGCCGGCGTTAACATCGCGGGTT
TTGACTTTGGCTGTACCACAGATGGCACTTGCGTTACCTCGAAGGTTTAT
CCTCCGTTGAAGAACTTCACCGGCTCAAACAACTACCCCGATGGCATCGG
CCAGATGCAGCACTTCGTCAACGAGGACGGGATGACTATTTTCCGCTTAC
CTGTCGGATGGCAGTACCTCGTCAACAACAATTTGGGCGGCAATCTTGAT
TCCACGAGCATTTCCAAGTATGATCAGCTTGTTCAGGGGTGCCTGTCTCT
GGGCGCATACTGCATCGTTGACATCCACAATTATGCTCGATGGAACGGTG
GGATCATTGGTCAGGGCGGCCCTACTAATGCTCAATTCACGAGCCTTTGG
TCGCAGTTGGCATCAAAGTACGCATCTCAGTCGAGGGTGTGGTTCGGCAT
CATGAATGAGCCCCACGACGTGAACATCAACACCTGGGCTGCCACGGTCC
AAGAGGTTGTAACCGCAATCCGCAACGCTGGTGCTACGTCGCAATTCATC
TCTTTGCCTGGAAATGATTGGCAATCTGCTGGGGCTTTCATATCCGATGG
CAGTGCAGCCGCCCTGTCTCAAGTCACGAACCCGGATGGGTCAACAACGA
ATCTGATTTTTGACGTGCACAAATACTTGGACTCAGACAACTCCGGTACT
CACGCCGAATGTACTACAAATAACATTGACGGCGCCTTTTCTCCGCTTGC
CACTTGGCTCCGACAGAACAATCGCCAGGCTATCCTGACAGAAACCGGTG
GTGGCAACGTTCAGTCCTGCATACAAGACATGTGCCAGCAAATCCAATAT
CTCAACCAGAACTCAGATGTCTATCTTGGCTATGTTGGTTGGGGTGCCGG
ATCATTTGATAGCACGTATGTCCTGACGGAAACACCGACTGGCAGTGGTA
ACTCATGGACGGACACATCCTTGGTCAGCTCGTGTCTCGCAAGAAAGGGA
TCCTAAGGTACC

Where nucleotides:
1 to 6 is a XbaI restriction site;
7 to 1197 is the mature endoglucanase from T. reesei (GenBank accession numbers DQ 178347 or P07982), with the following nucleotide changes introduced (numbering according to the DQ178347 DNA sequence): G267T and C576T;
1198 to 1203 is a BamHI restriction site;
1204 to 1206 is a TAA STOP codon; and
1207 to 1212 is a KpnI restriction site.

Each of the above plasmids was used to create corresponding expression plasmids for cell wall attached cellulases. For cell wall attached CBHII, pUC119-AF101 DNA was digested with HindIII-EcoRI and the ˜3370 by DNA fragment was gel purified. The purified DNA fragment was ligated into the HindIII-EcoRI digested vectors YEplac112, YEplac181 and YEplac195, to generate YEplac112-AF101-at, YEplac181-AF101-at and YEplac195-AF 101-at, respectively. For cell wall attached BGLI, pUC119-AF102 DNA was digested with XbaI-BamHI and the ˜2520 by DNA fragment was gel purified. The purified DNA fragment was ligated into the XbaI-BamHI digested YEplac181-AF101-at vector, to generate YEplac181-AF102-at. For cell wall attached EGII, pUC119-AF103 DNA was digested with XbaI-BamHI and the ˜1212 by DNA fragment was gel purified. The purified DNA fragment was ligated into the XbaI-BamHI digested YEplac112-AF101-at vector, to generate YEplac112-AF103-at.

Expression plasmids for secreted cellulases were also generated. For secreted BGLI, pUC119-AF102 DNA was digested with XbaI-KpnI and the ˜2530 by DNA fragment was gel purified. The purified DNA fragment was ligated into XbaI-KpnI digested vectors YEplac181-AF101-at and YEplac195-AF101, to generate YEplac181-AF102-sec and YEplac195-AF102-sec, respectively. For secreted EGII, pUC119-AF103 DNA was digested with XbaI-KpnI and the ˜1212 by DNA fragment was gel purified. The purified DNA fragment was ligated into the XbaI-KpnI digested YEplac112-AF103-at vector, to generate YEplac112-AF103-sec. For secreted CBHII, pUC119-AF101 DNA was digested with XbaI-BamHI and the ˜1341 by DNA fragment was gel purified. The purified DNA fragment was ligated into the XbaI-BamHI digested YEplac195-AF102-sec, to generate YEplac195-AF101-sec.

### Example 2

### Construction of Expression Plasmids Encoding Butanol Pathway Genes

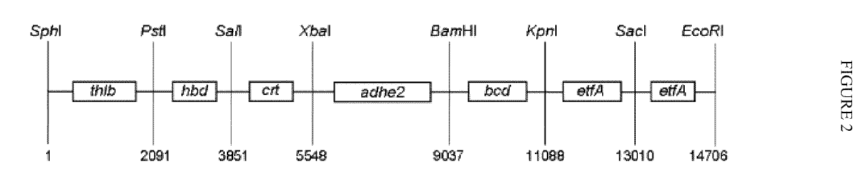

To express the butanol biosynthetic pathway (FIG. 1) in yeast, the AF 104 DNA was commercially synthesized by Blue Heron Bio, with the order and the position of the C. acetobutylicum genes in the AF 104 DNA shown in Table 1 and FIG. 2. The AF 104 DNA was cloned into the PENTR223 plasmid, which confers spectinomycin resistance to bacterial cells. To facilitate subsequent cloning, several restriction sites were removed from coding sequences of the C. acetobutylicum genes via one nucleotide substitutions that did not change the amino acid sequences. Specifically, the recognition sites for the restriction endonucleases shown below were mutated in the AF104 DNA as follows: XbaI (TCT/AAGA, 1014-1019), EcoRV (GA/TTATC, 1120-1125), PstI (CT/AGCAG, 1417-1422), PstI (CT/AGCAG, 6650-6655), EcoRI (GAAT/CTC, 6966-6971), KpnI (GGT/AACC, 7999-8004), EcoRV (8761-8766), EcoRI (GA/TATTC, 9850-9855), EcoRV (GATATC/T, 12380-12385). The AF104_PENTR223 plasmid does not contain sequences essential for replication of plasmid DNA in yeast, the yeast origin of replication was subcloned into AF104_PENTR223. Specifically, AF104_PENTR223 plasmid DNA was linearized by EcoRV digestion. The high copy (YEplac195, YEPlac112, YEplacl81) and low copy (YCplac33, YCplac 22 and YCplac 111) number bacterial-yeast shuttle vectors were digested with AatII/NarI and incubated with T4 DNA polymerase to blunt 5′- and 3′-protruding ends generated by the restriction digestion. The yeast DNA fragments of these plasmids containing yeast origins of replication were ligated to AF104_PENTR223. The resulting recombinant plasmids (Table 2) were able to grow on minimal media and expressed at least two enzymes responsible for butanol biosynthesis (see Example 8 below). As a quality control, plasmid DNAs were recovered from yeast cells, reintroduced into bacteria, purified and subjected to thorough restriction analysis. Remarkably, only two of fifty plasmid DNAs had an altered restriction map demonstrating that AF 104 DNA-derived plasmids are stable in yeast.

### Example 3

### Transformation of S. Cerevisiae and Transformant Selection

The derivatives of yeast strains AFY1 (MATα his3-Δ200 leu2-3,112 ura3-52 lys2-801 trp1-1) and AFY2 (MATa his3-Δ200 leu2-3,112 ura3-52 lys2-801 trp1-1) (Table 2) were used. These strains can be transformed with up to five plasmids carrying different selection markers. Transformation with the expression plasmids were performed with a lithium acetate method. Co-transformation with up to 3 plasmids was performed and the Trp⁺Ura⁺Leu⁺ colonies containing plasmids encoding cellulases or cellulases and butanol pathway genes were selected. To express the butanol pathway genes alone, single drop-out media were used.

The yeast transformation procedure used was a slightly modified version of the protocol described in Ausubel et al., (2002). Cells from an overnight culture were resuspended in 50 mL YPD (start OD₆₀₀ of 0.2) and grown to an OD₆₀₀ of 0.5-0.7. The cells were harvested by centrifugation (1,500 g, 5 min) and resuspended in 20 mL sterile distilled water. The cells were harvested by centrifugation and resuspended in 1.5 mL of freshly prepared sterile TE/LiOAc (prepared from 10× concentrated stocks; 10× TE-0.1 M Tris-HCl, 0.01 M EDTA, pH 7.5; 10× LiOAc-1 M LiOAc adjusted to pH 7.5 with dilute acetic acid). For a gene disruption experiment, ˜5 μg disruption cassette DNA was mixed with 70 μg of freshly denatured salmon sperm DNA (10 mg/mL, boiled for 20 min in a water bath, then chilled in ice/water) and 200 μL cells in TE/LiOAc were added and carefully mixed. Immediately, 1,200 μg of freshly prepared sterile 40% PEG 4,000 (prepared from stock solutions: 50% PEG 4000, 10×TE, 10× LiOAc, 8:1:1 v/v, pH 7.5) were added and carefully mixed. Cells were incubated for 30 min at 30° C. with constant agitation. Cells were incubated for 15 min at 42° C. and then collected by centrifugation (4,000 g, 1 min). Cells were resuspended in 200 μl YPD and plated onto selective plates. Plates were incubated at 30° C. until colonies appeared.

### Example 4

### Cellulose Treatment

All chemicals, media components and supplements were of analytical grade standard. Phosphoric acid-swollen cellulose (PASC) was prepared as described by Den Haan et al., (2007). Briefly, Avicel® PH-101 (Fluka) (2 g) was first soaked with 6 mL of distilled water. Then, 50 mL of 86.2% phosphoric acid was added slowly to the tube and mixed well, followed by another 50 mL of phosphoric acid and mixing. The transparent solution was kept at 4° C. overnight to completely solubilize the cellulose, until no lumps remained in the reaction mixture. Next, 200 mL of ice-cold distilled water was added to the tube and mixed, followed by another 200 mL of water and mixing. The mixture was centrifuged at 3,500 rpm for 15 min and the supernatant was removed. Addition of distilled water and subsequent centrifugation were repeated. Finally, 10 mL of 2M sodium carbonate and 450 mL of water were added to the cellulose, followed by 2 or 3 washes with distilled water, until a final pH of 5-7 was obtained. Acid treatment of Whatman® Paper #1 was done as described above for Avicel®, except only 1 g of shredded paper was used.

### Example 5

### Yeast Fermentation

Single colonies were inoculated into 10 mL of media with appropriate supplements and with 2% glucose as a carbon source and incubated aerobically for 24-72 hours at 30° C. Yeast cells were collected by centrifugation for 10 min at 4,000 rpm and resuspended in 100 mL of media with 2% glucose. After incubation under aerobic conditions for 24-72 hours at 30° C. cells were harvested by centrifugation and washed with distilled water twice. Cell pellets were inoculated in 10 mL of media with either 2% glucose, or 40% PASC or 40% treated Whatman® Paper and butanol or ethanol fermentations were anaerobically performed at 30° C. in 15 mL tubes with closed caps. 0.2 mL aliquots were collected at different time points and analyzed using gas chromatography for butanol and ethanol concentration.

### Example 6

### Gene Disruption Using the loxP-his5-loxP Disruption Cassettes

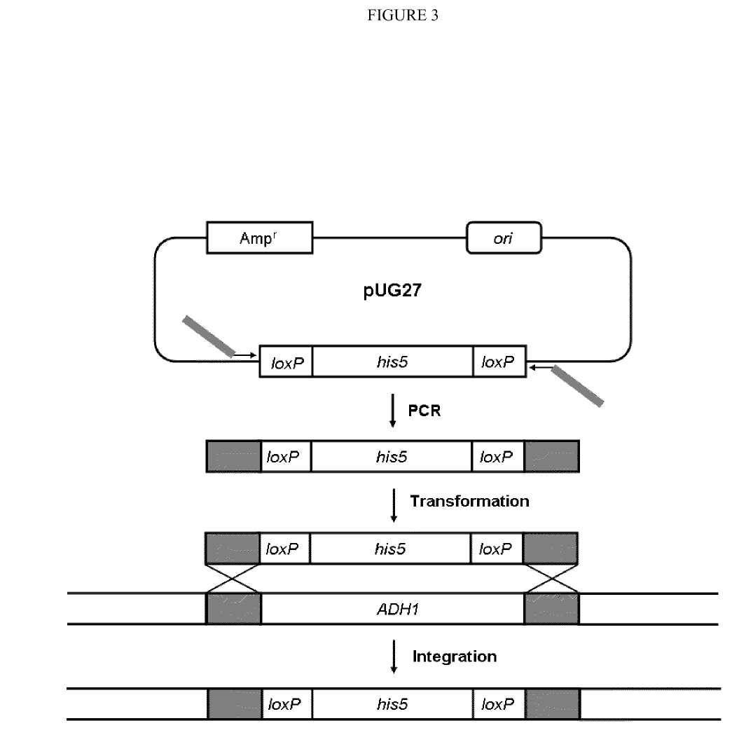

S. cerevisiae is a very efficient ethanol producer. Therefore, to avoid competition between ethanol and butanol biosynthetic pathways, the ADH1 and ADH5 genes in the laboratory strains AFY1 and AFY3 were deleted using standard techniques. The chromosomal ADH1 and ADH5 genes were inactivated by the PCR-based gene deletion using the pUG27 plasmid (Gueldener et al. 1996) as a PCR template to create a DNA fragment that directed replacement of the chromosomal ORFs with the Schizosachharomyces pombe his5 gene by homologous recombination in diploid yeast cells. Two cassettes were amplified using ADH1 and ADH5 disruption primers (Table 3). The 5′-50 nucleotides of the primers are homologous to target gene sequences upstream of the ATG start codon and downstream of the termination codon, respectively. The 3′-segments are homologous to sequences to the right and to the left of loxP motifs of the disruption cassettes (FIG. 3).

Importantly, deletion of the ADH1 gene led to significant decrease of ethanol biosynthesis. Double mutant strains including mutation in the adh1 and adh5 genes were also constructed. The S. cerevisiae genome encodes 8 alcohol dehyrodenases, at least 4 of which are involved in ethanol production. Therefore, inactivation of the corresponding genes can result in blocking ethanol synthesis and may significantly increase butanol production.

To confirm correct integration of the disruption cassettes into the ADH1 and ADH5 loci, diagnostic PCR was performed on the His⁺ transformants using a combination of corresponding target gene-specific primers (A, D) and disruption cassette specific primers (B, C) (Table 3). The heterozygous diploids were sporulated, and tetrads were dissected.

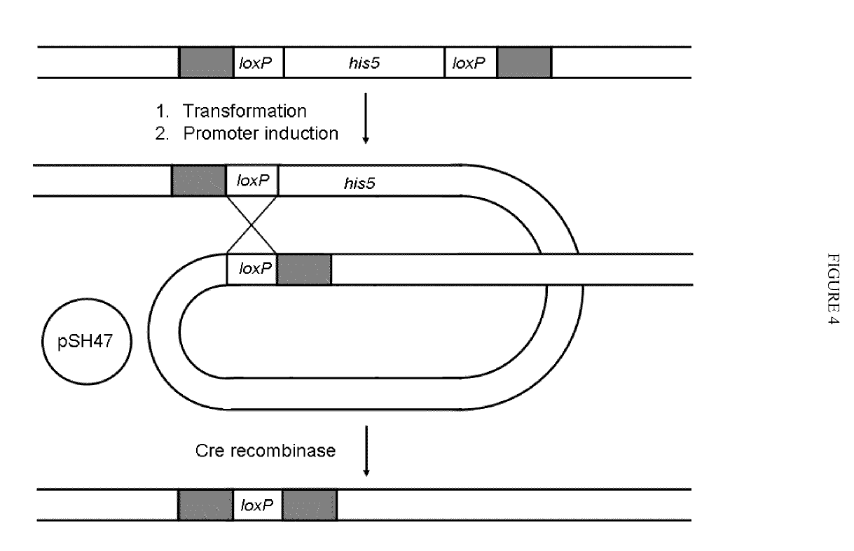

To use the his5 marker repeatedly for several gene disruptions in one strain, it is necessary to eliminate the marker from the successfully disrupted gene. The adh1 and adh5 mutant strains, in which corresponding genes were disrupted by the loxP-his5-loxP cassettes, were transformed with the cre expression plasmid pSH47 that carries the URA3 marker gene and the cre gene under the control of the inducible GAL1 promoter (Guldener at al., 1996)(FIG. 4). Expression of the Cre recombinase was induced by shifting cells from glucose to galactose medium and incubating for 2 hours in the galactose medium. Cells that lost the his5 marker gene were detected by replica plating yeast colonies on minimal glucose-containing plates without histidine. Loss of the his5 marker gene was verified by diagnostic PCR. The Cre expression plasmid was removed from these strains by streaking cells on plates containing 5-fluoroorotic acid to counterselect for the loss of the plasmid.

### Example 7

### Preparation of Protein Extracts from Yeast

Yeast cell-free extracts were prepared essentially as described in Ausubel et al., (2002). Overnight yeast cultures were diluted to an OD₆₀₀ of 0.2 and then grown to an OD₆₀₀ of 0.8-1.0 in 10 mL of selective minimal media. Cells were harvested by centrifugation and resuspended in 200 μL of glass beads disruption buffer containing protease inhibitors (20 mM Tris-HCl, pH 7.9; 10 mM MgCl₂; 1 mM EDTA, 1 mM dithiothreitol, 5% glycerol, 0.3 M ammonium sulfate; 1 μg/mL leupeptin, antipain, chimostatin, pepstatin and aprotinin). An equal volume of chilled acid-washed glass beads was added and the suspensions were vortexed at maximum speed for 1 min at 4° C. Tubes were placed on ice for 2 min and vortexed again 4 more times. The aqueous phase was collected and kept on ice. Glass beads were washed with 2 volumes of glass beads disruption buffer. Pooled cell free extracts were centrifuged for 15 minutes at 12,000 g, 4° C. and stored at −80° C.

### Example 8

### Enzyme Assays

All enzyme assays are performed at 25° C.

Using acetoacetyl-Co and CoA as substrates, THL activity was determined from the decrease in acetoacetyl-CoA concentration as measured at 303 nm (Wiesenborn et al., 1988) using a Genesys 10 UV/Visible spectrophotometer (Thermo Scientific, Waltham, Mass.). To start the enzymatic reaction, cell extracts (10 μL) were added to a solution containing 100 mM Tris-HCl (pH 8.0), 10 mM MgCl₂, 1 mM dithiothreitol, 50 μM acetoacetyl-CoA, and 0.2 mM CoA. The decrease in absorbance was monitored in the sample solution and a control solution, from which CoA was omitted.

HBD activity was measured at 345 nm by monitoring the decrease in NADH concentration resulting from β-hydroxybutyryl-CoA formation from acetoacetyl-CoA (Hartmanis and Gatenbeck, 1984). Cell extracts were added to a mixture containing 100 mM MOPS (pH 7.0), 1 mM dithiothreitol, 0.1 mM acetoacetyl-CoA and 0.15 mM NADH. Acetoacetyl-CoA was omitted in controls.

CRT activity is measured by monitoring the decrease in crotonyl-CoA concentration at 263 nm resulting from β-hydroxybutyryl-CoA formation from crotonyl-CoA (Hartmanis and Gatenbeck, 1984). Cell extracts are added to a mixture containing 100 mM Tris-HCl (pH 7.6) and 50 μM crotonyl-CoA.

The cell extracts for BCD assays are prepared as described above in an anaerobic chamber filled 95% N₂ and 5% H₂. BCD activity is assayed by monitoring at 300 nm the ferricenium ion, which acts as an electron donor during butyryl-CoA formation from crotonyl-CoA, (Lehman et al., 1990). To a mixture containing cell extract and 50 mM MOPS (pH 7.0), crotonyl-CoA is added to 0.4 mM, and following 10 min equilibration, ferricenium ion is added to a final concentration of 0.2 mM. The decrease in the absorbance of the sample solution and a control solution without crotonyl-CoA is monitored.

To measure BYDH and BDH activities, aerobically grown cultures are incubated under anaerobic condition for 3 hours with gentle stirring and the cell extract is then prepared in an anaerobic chamber. The BYDH activity assay is performed using yeast alcohol dehydrogenase (Dürre et al. 1987). In this coupled assay, BYDH converts butyryl-CoA to butyraldehyde, which is further converted to butanol by the alcohol dehydrogenase resulting in consumption of 2 NADH molecules. The mixture containing cell extract, 50 mM MES buffer (pH 6.0), 100 mM KCl, 0.15 mM NADH and 3 U of yeast-derived alcohol dehydrogenase is incubated for 10 min and 0.2 mM butyryl-CoA is then added to the mixture. The decrease in NADH concentration is measured at 345 nm. Butyryl-CoA is omitted from controls.

BDH activity is measured by monitoring the decrease in NADH concentration at 345 nm resulting from butanol formation from butyraldehyde in a sample solution and a control solution without butyraldehyde (Dürre et al. 1987). The reaction mixture containing cell extract, 50 mM MES (pH 6.0) and 0.15 mM NADH is incubated for 10 min prior to addition of 35 mM butyraldehyde.

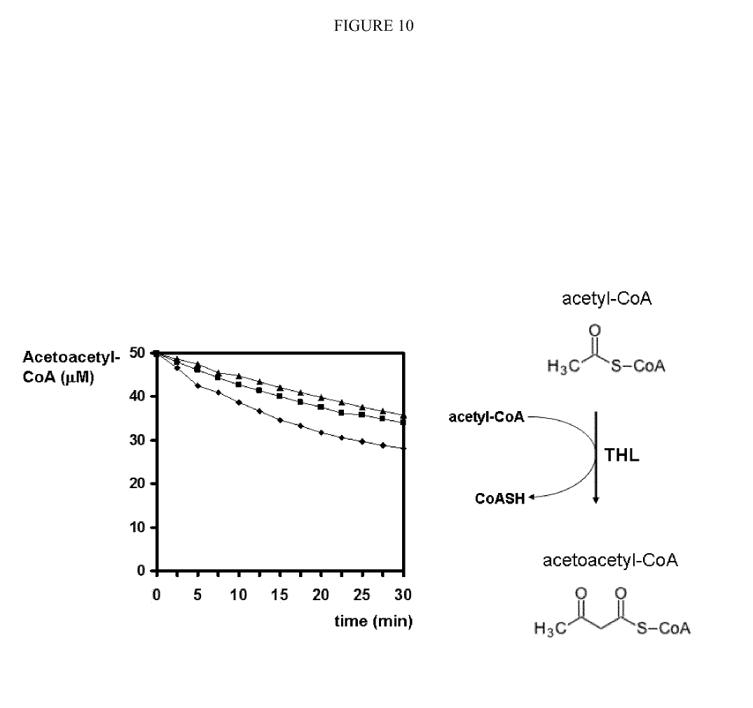

To date, the activities of two C. acetobutylicum enzymes responsible for biosynthesis of butanol in recombinant yeast cells transformed with AF 104 derivatives were tested as described above. As a result of acetyl-CoA acetyltransferase (thiolase, THL) activity, 2 acetyl-CoA molecules form from acetoacetyl-CoA and CoA. Transformation of AFY10 yeast strains with a high copy plasmid expressing the butanol pathway genes significantly accelerated decrease in acetoacetyl-CoA concentration in vitro (FIG. 10). For example, after 30 min incubation only 56% of acetoacetyl-CoA remained in the reaction mixture. By contrast, extracts prepared from cells transformed with vector DNA converted only 32% of the substrate.

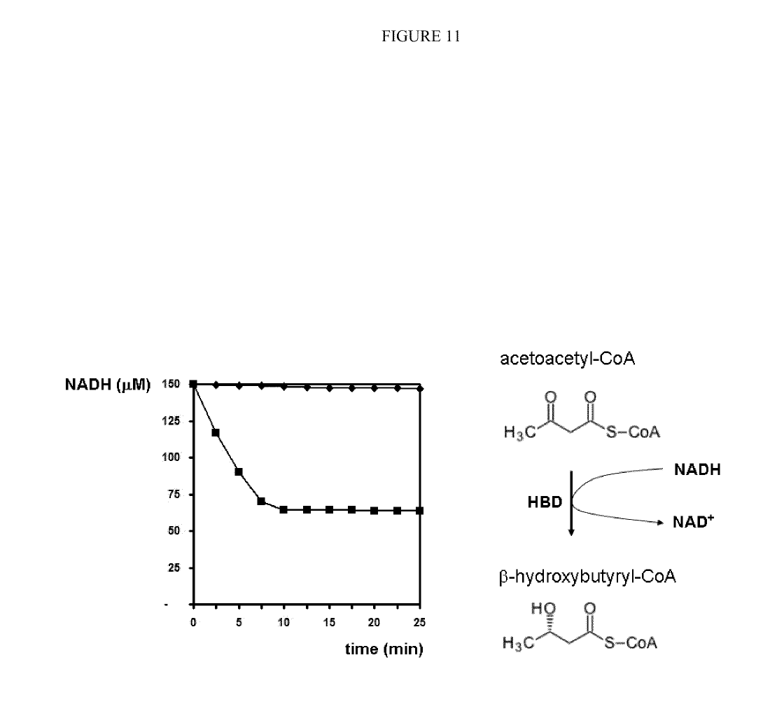

β-hydroxybutyryl-CoA dehydrogenase (HBD) activity involves formation of β-hydroxybutyryl from acetoacetyl-CoA in an NADH coupled reaction. Incubation of the substrate with protein extracts prepared from yeast cells transformed with vector DNA alone did not lead to significant decrease in NADH concentration (98% NADH remained in the reaction mixture after 25 min incubation) (FIG. 11). However, plasmid DNAs encoding the butanol pathway resulted in a dramatic decrease in NADH concentration. After 10 min of incubation almost 50% of NADH was converted to NAD⁺.

### Example 9

### Gas Chromatography Analysis

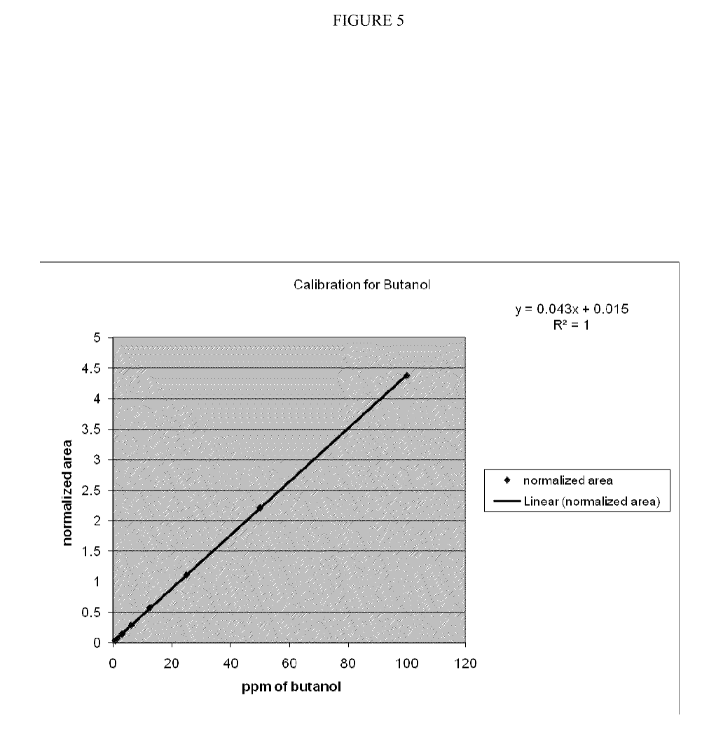

Fermentation products (e.g., ethanol and butanol) were analyzed using gas chromatography (GC) (5890 Series II Agilent Technologies, Wilmington, Del.) provided with a RTX-5 capillary column (30 m×0.53 mm i.d.×1.5 μm) (Restek, Bellefonte, Pa.) and flame ionization detection. Prior to analysis, the samples were centrifuged at 14,000× rpm for 10 minutes. The samples were diluted 20-fold with a 25 ppm aqueous solution of n-propanol as an internal standard. Helium was used as a carrier gas at 5 mL/min and was split 1 to 20 before the capillary column. The column was heated to 40° C. for 4 minutes and then ramped to 130° C. at a rate of 30° C./min. The GC was equipped with a 7673B auto-sampler (Agilent Technologies) and data were collected through contact closures and analyzed using Peak Simple software (SRI Instruments Torrance, Calif.). Linear calibration curves were developed for ethanol and butanol covering the ranges of 1000 ppm to 0.8 ppm and 100 ppm to 0.8 ppm, respectively. FIG. 5 is an example of a calibration curve for butanol.

### Example 10

### Fermentation Butanol and Ethanol from Cellulose by Recombinant Yeast

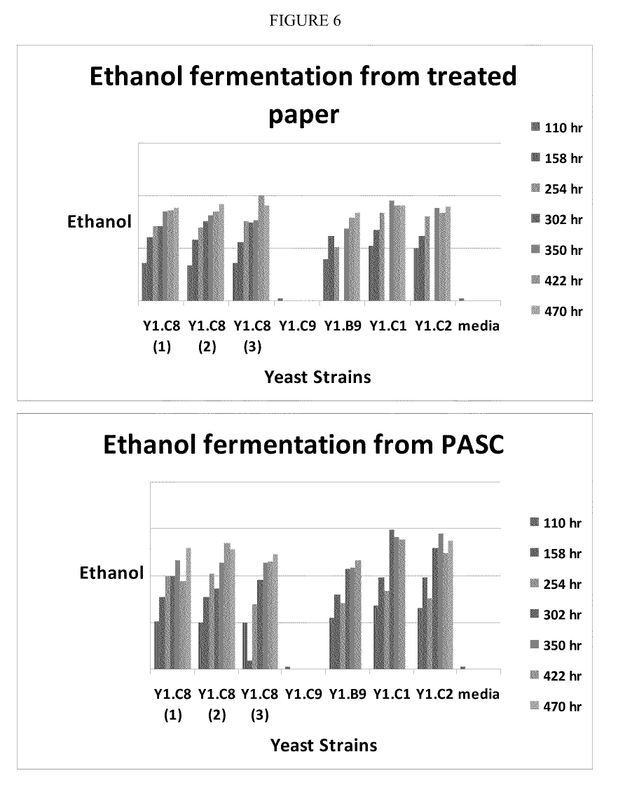

Several yeast strains were constructed for production of butanol and ethanol from cellulose. To ferment cellulose to butanol and ethanol, strains were constructed that codisplay three cellulases (EGII, CHBII and BGLI) on the yeast cell wall surface. Furthermore, a second set of strains that produce secreted forms of the same cellulases were developed. The strains with surface displayed cellulases and the strains expressing secreted cellulases are efficient hosts for the production of ethanol from either PASC or treated paper (FIG. 6). FIG. 6 illustrates fermentation of cellulose to ethanol by the above yeast strains. Fermentations were performed in 15 mL tubes with 10 mL of minimal media and 40% PASC or treated Whatman® Paper. PASC, an amorphous type of cellulose, was prepared from Avicel® by treatment with 85% phosphoric acid. Avicel® is a commercially available, crystalline form of cellulose produced by acid reflux hydrolysis of wood. Several independent recombinant yeast strains were used for each fermentation experiment. Yeast strains transformed with empty vectors, i.e., without cellulases genes, were used as negative controls. Remarkably, the ethanol producing yeast strains depolymerized cellulose and fermented it to ethanol with almost 100% of the maximum theoretical yield and produced more than 4 gram per liter of ethanol.

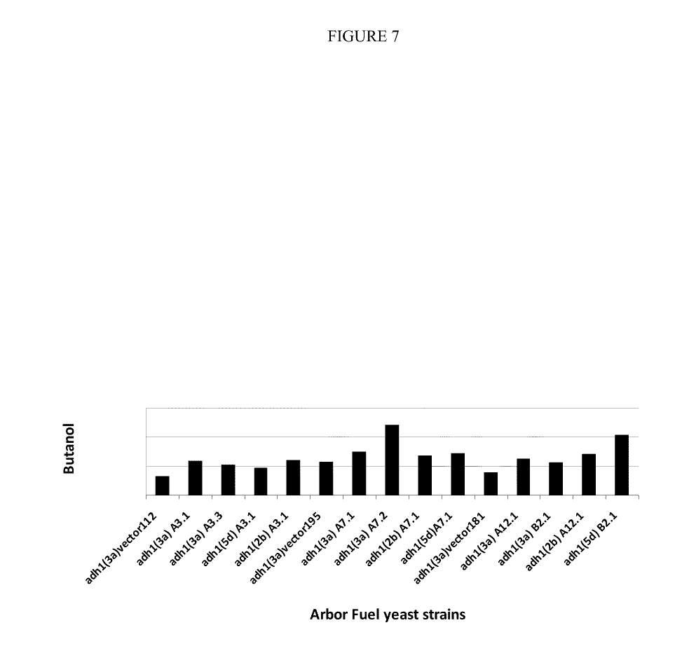

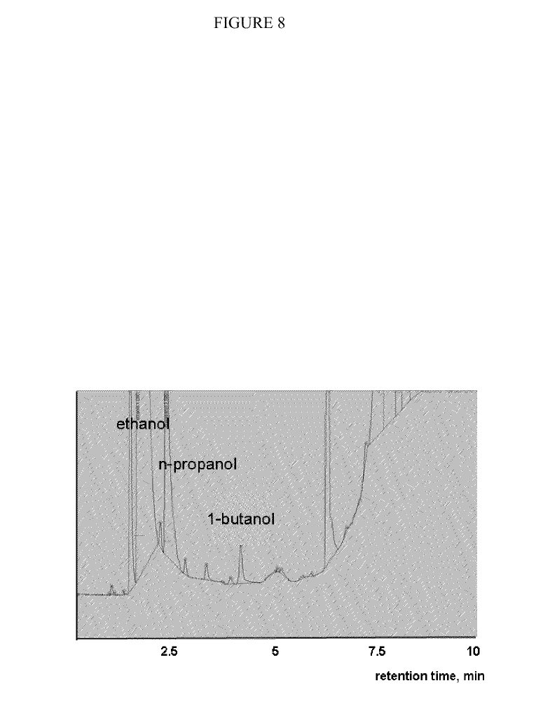

To ferment glucose to butanol, yeast strains were constructed that express the enzymes from the butanol pathway of FIG. 1. These strains were used for butanol fermentation from 2% glucose. Butanol fermentations were done under anaerobic conditions using a GasPak™ EX Anaerobic Generating System. This system offers waterless anaerobic conditions with 4-10% carbon dioxide and ˜0.1% oxygen. FIG. 7 shows butanol fermentation from glucose with twelve yeast strains containing butanol pathway genes. Three vector controls were used as negative controls. One yeast strain, i.e., adh1(3a)A7.2, produced more than 0.018 g/L of butanol, as measured by gas chromatography (FIG. 8). It should be noted that the fermentation experiments were conducted in yeast strains in which only one enzyme involved in the final stage of ethanol production, Adh1, was inactivated. As the S. cerevisiae genome encodes 8 alcohol dehyrodenases, at least 4 of which are involved in ethanol production, it is expected that butanol yield in yeast strains bearing multiple adh mutations will be significantly higher.

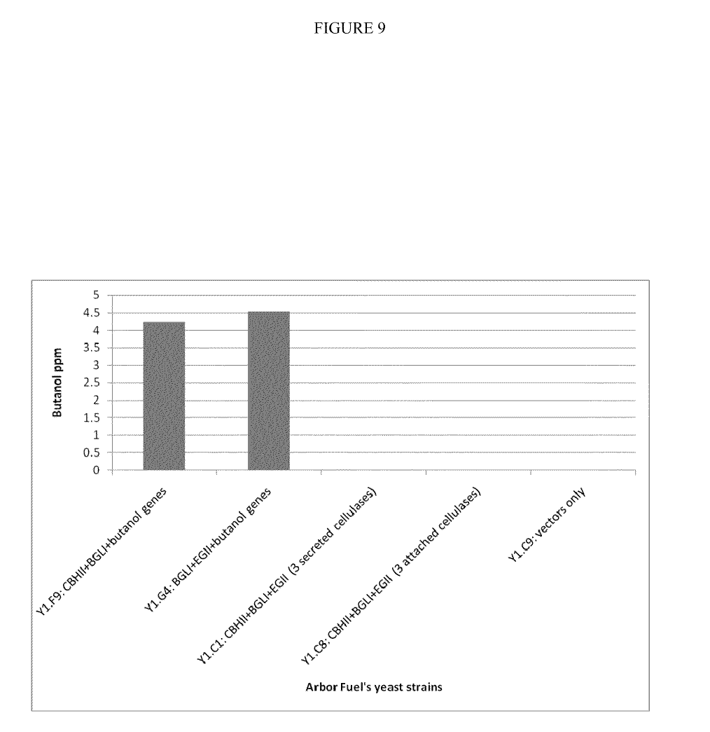

To ferment cellulose to butanol, yeast strains were constructed that express all enzymes from the butanol pathway and two secreted cellulases: EGII and CBHII; EGII and BGLI; or CBHII and BGLI. These strains were used for butanol fermentation from 40% PASC. Butanol fermentations were done under anaerobic conditions using a GasPak™ EX Anaerobic Generating System. FIG. 9 shows butanol fermentation from cellulose with several of Arbor Fuel's yeast strains containing butanol pathway and cellulase genes. One yeast strain Y1.F9 containing CBHII and BGLI produced 4.3 ppm, while another strain Y1.G4 containing EGII and BGLI produced 4.8 ppm of butanol.

### Example 11

### Sensitivity of Laboratory and Industrial Yeast Strains to Butanol

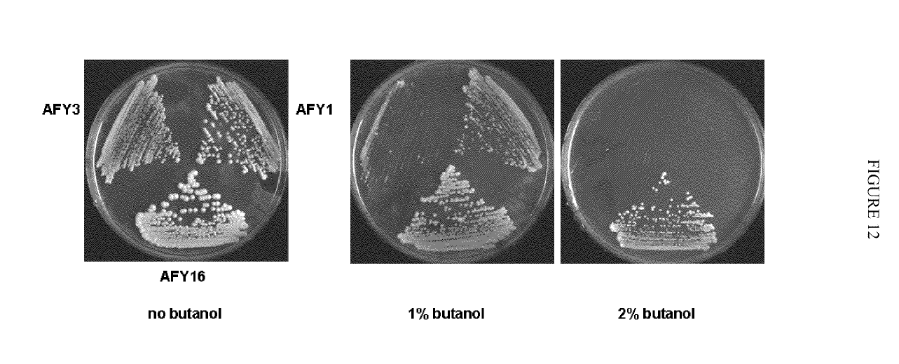

To produce butanol at industrial levels, host cells that tolerate high butanol concentration are preferable. The sensitivity of laboratory and industrial yeast strains to butanol was tested. Growth of both laboratory strains tested (AFY1, AFY3) was severely compromised on plates containing 1% butanol. By contrast, the industrial yeast strain AFY16, which is a wild type polyploid yeast strain, tolerated up to 2% butanol without significant affect on growth rate (FIG. 12), suggesting the suitability of the AFY16 yeast strain and its derivatives for industrial butanol production.

### Example 12

### Expression of Laccase in S. Cerevisiae

Laccase can be used for enzymatic detoxification of lignocellulosic hydrolysates. A S. cerevisiae strain with enhanced resistance to phenolic inhibitors, and thereby improved ability to ferment lignocellulosic hydrolysates, is obtained by heterologous expression of laccase. The yeast S. cerevisiae can be used to ferment the sugars in lignocellulose hydrolysates. A problem associated with the fermentation process is the presence of inhibitors in the lignocellulose hydrolysate. Inhibitors may include phenolic compounds, furan derivatives, aliphatic acids and extractives. There are several different methods for detoxification of lignocellulose hydrolysates prior to fermentation (Olsson and Hahn-Hagerdal, 1996). An enzymatic detoxification method, using laccase from T. versicolor, was recently developed (Jonsson et al., 1998). Laccase specifically removed the phenolic compounds without changing the concentrations of furan derivatives, aliphatic acids and fermentable sugars. Enzymatic detoxification methods allow the construction of S. cerevisiae strains that are more resistant to fermentation inhibitors. Introduction of cellulase genes into these strains, convert these naturally non-cellulollytic yeast into microorganisms that enable growth and fermentation on pretreated lignocelluloses. The laccase expression construct is similar to the cellulase constructs. The cloning of the laccase gene can be done as described in Example 1 for the cloning of cellulases. Briefly, the mature laccase PDXA 1 b (AJ005018) from Pleurotus ostreatus is fused with the secretion signal sequence of glucoamylase (D00049) from R. oryzae. The secretion signal is responsible for delivery of laccase to the cell wall and secretion outside the cell. The P. ostreatus laccase expression construct can be coexpressed with the expression constructs for endoglucanase II and cellobiohydrolase II from T. reesei, and A. aculeatus (β-glucosidase.

### Example 13

### Expression of Xylose Assimilation Enzymes in S. Cerevisiae

The purpose of this Example is to describe how xylose fermenting S. cerevisiae strains can be engineered. Wild-type strains of S. cerevisiae cannot utilize pentoses, such as xylose. However efficient fermentation of pentose sugars is necessary to attain economically feasible processes for ethanol and butanol production from lignocellulosic biomass. Anaerobic xylose fermentation by S. cerevisiae was first demonstrated by heterologous expression of xylose reductase (XR) and xylitol dehydrogenase (XDH) from Pichia stipitis together with overexpression of the endogenous xylulokinase (XK) (Ho et al., 1998, 1999). Alcohol fermentation from xylose was also performed by a recombinant S. cerevisiae strain carrying only one heterologous xylose isomerase (Xi) gene from the fungus Piromyces sp. (Kuyper et al., 2003). The open reading frame encoding XI (GenBank accession number AJ249909) will be synthesized by Blue Heron Bio. Sites for restriction endonucleases SalI and KpnI will be introduced at 5′- and 3′-ends of DNA, respectively. The sites for restriction endonucleases HindIII and KpnI will be changed via one nucleotide substitutions that do not change the amino acid sequences. The resulting plasmid, pUC119-AF105, will be digested with SalI-KpnII and the ˜1326 by DNA fragment will be gel purified. The purified DNA fragment will be ligated into the SalI-KpnI digested vector YEplac195-AF101-at to generate plasmid pYEplac195-AF105. This plasmid will be used for the transformation of yeast cells as well as for cotransformation of cells already containing cellulase genes and butanol pathway genes as described above.

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

TABLE 1
The butanol biosynthetic pathway genes

| | Gene bank | Position in | | Number of | |
|---|---|---|---|---|---|
| | accession | AF104 | | amino | EC |
| Gene name | number | DNA | Enzyme name | acids | number |
| Thlb | AF072735/ | 660-1838 | Acetyl-CoA | 392 | 2.3.1.9 |
| | AE001437.1 | | acetyltransferase | | |
| | | | (Thiolase, THL) | | |
| Hbd | AE001437.1 | 2750-3598 | 3-Hydroxybutyryl-CoA | 282 | 1.1.1.157 |
| | | | dehydrogenase (HBD) | | |
| Crt | U17110.1/ | 4510-5295 | 3-Hydroxybutyryl-CoA | 261 | 4.2.1.55 |
| | AE001437.1 | | dehydratase (Crotonase, | | |
| | | | CRT) | | |
| adhe2 | AF321779/ | 6208-8784 | Aldehyde-alcohol | 858 | |
| | AE001437.1 | | dehydrogenase | | |
| | | | (AADH2, BYDH, BDH) | | |
| Bcd | AE001437.1 | 9696-10835 | Butyryl-CoA | 379 | 1.3.99.2 |
| | | | dehydrogenase (BCD) | | |
| etfA | AE001437.1 | 11747-12757 | Electron-transfer | 336 | NA |
| | | | flavoprotein α subunit | | |
| | | | (ETFα) | | |
| etfB | AE001437.1 | 13669-14458 | Electron-transfer | 259 | NA |
| | | | flavoprotein β subunit | | |
| | | | (ETFβ) | | |

TABLE 2
Yeast strains and plasmids used
Yeast strains

| | |
|---|---|
| AFY1 | MATα his3-Δ200 leu_3,112 ura3-52 lys2-801 trp1-1 |
| AFY2 | MATa his3-Δ200 leu_3,112 ura3-52 lys2-801 trp1-1 |
| AFY3 | MATα/a his3-Δ200 leu_3,112 ura3-52 lys2-801 trp1-1 |
| AFY10 | MATα his3-Δ200:: leu_3,112 ura3-52 lys2-801 trp1-1 adh1-Δ1::his5⁺ |
| AFY19 | MATα his3-Δ200:: leu 3,112 ura3-52 lys2-801 trp1-1 adh5-Δ1::his5⁺ |
| AFY28 | MATα his3-Δ200:: leu_3,112 ura3-52 lys2-801 trp1-1 adh1-Δ1::his5⁺ adh5-Δ1::his5⁺ |

Plasmids

| | |
|---|---|
| AF104_PENTR223 | The AF104 DNA cloned into PENTR223 vector conferring |
| | resistance to spectinomycin |
| pAF104/112A3 | The AF104_PENTR223 containing the AatII/NarI fragment of |
| | YEplac112 encoding the yeast 2μ origin of replication |
| pAF104/195A7 | The AF104_PENTR223 containing the AatII/NarI fragment of |
| | YEplac195 encoding the yeast 2μ origin of replication |
| pAF104/181A12 | The AF104_PENTR223 containing the AatII/NarI fragment of |
| pAF104/181B2 | YEplac181 encoding the yeast 2μ origin of replication |
| pAF104/22 | The AF104_PENTR223 containing the AatII/NarI fragment of |
| | YCplac22 encoding the yeast CEN4 origin of replication |
| pAF104/339 | The AF104_PENTR223 containing the AatII/NarI fragment of |
| | YCplac33 encoding the yeast CEN4 origin of replication |
| pAF104/11116 | The AF104_PENTR223 containing the AatII/NarI fragment of |
| | YCplac111 encoding the yeast CEN4 origin of replication |
| pUC119-AF101 | cellobiohydrolase II (CBHII) construct |
| YEplac112-AF101-at | expression construct with attached CBHII |
| YEplac181-AF101-at | expression construct with attached CBHII |
| YEplac195-AF101-at | expression construct with attached CBHII |
| YEplac181-AF102-at | expression construct with attached BGLI |
| YEplac112-AF103-at | expression construct with attached EGII |
| YEplac195-AF101-sec | expression construct with secreted CBHII |
| YEplac181-AF102-sec | expression construct with secreted BGLI |
| YEplac112-AF103-sec | expression construct with secreted EGII |

TABLE 3
List of oligonucleotides
Target gene/Disruption marker
Gene disruption primers

ADH1

(SEQ ID NO: 4)

GCACAATATTTCAAGCTATACCAAGCATACAATCAACTATCTCATATACA
cagctgaagcttcgtacgc

(SEQ ID NO: 5)

TTTTTTATAACTTATTTAATAATAAAAATCATAAATCATAAGAAATTCGC
gcataggccactagtggatctg
ADH5

(SEQ ID NO: 6)

AAGATACCTAAGAAAATTATTTAACTACATATCTACAAAATCAAAGCATC
cagctgaagcttcgtacgc

(SEQ ID NO: 7)

ATAGCTTATATAAAAAGTAAAAATATATTCATCAAATTCGTTACAAAAGA
gcataggccactagtggatctg

Verification primers/target gene-specific

| | | | |
|---|---|---|---|
| ADH1 | A | TCTCTCTCCCCCGTTGTTGT | (SEQ ID NO: 8) |
| | D | CTCAGGTAAGGGGCTAGTAG | (SEQ ID NO: 9) |
| ADH5 | A | GCGCCATTCAAGTCCCGCGA | (SEQ ID NO: 10) |
| | D | CAATTTAACCAATTTCTACTC | (SEQ ID NO: 11) |

Verification primers/disruption cassette
specific

| | | | |
|---|---|---|---|
| his5⁺ | kan-B | GGATGTATGGGCTAAATG | (SEQ ID NO: 12) |
| | kan-C | CCTCGACATCATCTGCCC | (SEQ ID NO: 13) |

### REFERENCES

Ausubel F M, Brent R, Kingston R E, More D D, Seidman J G, Smith J A, Struhl K. 2002. Sort Protocols in Molecular Biology. John Wiley and Sons, New York.
Den Haan R, Rose S H, Lynd L R, van Zyl W H. 2007. Hydrolysis and fermentation of amorphous cellulose by recombinant Saccharomyces cerevisiae. Metab Eng 9:87-94.
Durre P, Kuhn A, Gottwald M, Gottschalk. 1987. Enzymatic investigations on butanol dehydrogenase and butyraldehyde dehydrogenase in extracts of Clostridium acetobutylicum. Appl Microbiol Biotechnol 26:268-272.
Fujita Y, Ito J, Ueda M, Fukuda H, Kondo A. 2004. Synergistic saccharification, and direct fermentation to ethanol, of amorphous cellulose by use of an engineered yeast strain codisplaying three types of cellulolytic enzyme. Appl Environ Microbiol 70:1207-1212.
Guldener U, Heck S, Fiedler T, Beinhauer J, Heenmann J. 1996. A new efficient gene disruption cassette for repeated use in budding yeast. Nucleic Acid Research 24:2519-2524.
Hartmanis M G, Gatenbeck S. 1984. Intermediary Metabolism in Clostridium acetobutylicum: Levels of Enzymes Involved in the Formation of Acetate and Butyrate. Appl Environ Microbiol 47:1277-1283.
Ho N W, Chen Z, Brainard A P. 1998. Genetically engineered Saccharomyces yeast capable of effective cofermentation of glucose and xylose. Appl Environ Microbiol 64:1852-1859.
Ho N W, Chen Z, Brainard A P, Sedlak M. 1999. Successful design and development of genetically engineered Saccharomyces yeasts for effective co-fermentation of glucose and xylose from cellulosic biomass to fuel ethanol. Adv Biochem Eng Biotechnol 65:163-192.
Jonsson L J, Palmqvist E, Nilvebrant N O, Hahn-Hagerdal B. 1998. Detoxifcation of wood hydrolysates with laccase and peroxidase from the white-rot fungus Trametes versicolor. Appl Microbiol Biotechnol 49:691-697.
Kuyper M, Harhangi H R, Stave A K, Winkler A A, Jetten M S, de Laat W T, den Ridder J J, Op den Camp H J, van Dijken J P, Pronk J T. 2003. High level functional expression of a fungal xylose isomerase: the key to efficient ethanolic fermentation of xylose by Saccharomyces cerevisiae? FEMS Yeast Res 4:69-78.
Lehman T C, Hale D E, Bhala A, Thorpe C. 1990. An acyl-coenzyme A dehydrogenase assay utilizing the ferricenium ion. Anal Biochem 186:280-284.
Lynd L R, Weimer P J, van Zyl W H, Pretorius I S. 2002. Microbial cellulose utilization: fundamentals and biotechnology. Microbiol. Mol Biol Rev 66:506-577.
Olsson L, Hahn-Hagerdal B. 1996. Fermentation of lignocellulosic hydrolysates for ethanol production. Enzyme Microb Technol 18:312-331.
Sambrook J, Fritsch E F, Maniatis T. 1989. Molecular Cloning: A Laboratory Manual. Cold Spring Harbor Laboratory Press: Cold Spring Harbor, N.Y.
Sherman F, Fink G R, Hicks J B. 1986. Methods in Yeast Genetics. Cold Spring Harbor Laboratory Press: Cold Spring Harbor, N.Y.
van Zyl W H, Lynd L R, den Haan R, McBride J E. 2007. Consolidated bioprocessing for bioethanol production using Saccharomyces cerevisiae. Adv Biochem Eng Biotechnol 108:205-235
Wiesenborn D P, Rudolph F B, Papoutsakis E T. 1989. Coenzyme A transferase from Clostridium acetobutylicum ATCC 824 and its role in the uptake of acids. Appl Environ Microbiol 55:323-329.
Wolfenden R, Snider M J. 2001. The depth of chemical time and the powder of enzyme as catalysts. Acc Chem Res 34:938-945.
Zhang Y-H P, Lynd L R. 2004. Toward an aggregated understanding of enzymatic hydrolysis of cellulose: noncomplexed cellulose systems. Biotechnol Bioeng 88:797-824.
Zhang Y-H P, Himmel M E, Mielenz J R. 2006. Outook for cellulase improvement: screening and selection strategies. Biotechnol Adv 24:452-481

What is claimed is:

1. A recombinant microorganism, comprising:
(1) at least one heterologous butanol biosynthetic pathway gene that encodes a polypeptide that catalyzes a substrate to product conversion selected from the group consisting of:
(a) acetyl-CoA to acetoacetyl-CoA
(b) acetoacetyl-CoA to (S)-3-hydroxbutanoyl-CoA
(c) (S)-3-hydroxbutanoyl-CoA to crotonoyl-CoA
(d) crotonoyl-CoA to butyryl-CoA
(e) butyryl-CoA to butanal
(f) butanal to butanol; and
(2) at least one heterologous gene that encodes a cellulase enzyme;
wherein said recombinant microorganism converts cellulose to butanol.

2. The microorganism of claim 1, wherein said microorganism is a member of a genus selected from the group consisting of Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces.

3. The microorganism of claim 1, wherein said microorganism is a member of a species selected from the group consisting of Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis, Saccharomyces carlsburgenesis and Saccharomyces cerevisiae.

4. The microorganism of claim 2, wherein the microorganism is a Saccharomyces species.

5. The microorganism of claim 4, wherein the microorganism is a Saccharomyces cerevisiae.

6. The microorganism of claim 1, wherein the cellulase enzyme is selected from the group consisting of endoglucanase, exoglucanase and β-glucosidase.

7. The microorganism of claim 6, wherein the cellulase enzyme is selected from the group consisting of: endoglucanase II, cellobiohydrolase II, and β-glucosidase I.

8. The microorganism of claim 7, wherein the microorganism comprises heterologous genes that encode endoglucanase II, cellobiohydrolase II, and β-glucosidase I.

9. The microorganism of claim 8, wherein the endoglucanase II and cellobiohydrolase II genes are from T. reesei and the β-glucosidase I gene is from A. aculeatus.

10. The microorganism of claim 1, wherein the butanol biosynthetic pathway gene is selected from the group consisting of acetyl-CoA C-acetyltransferase (thiolase), 3-hydroxybutyryl-CoA dehydrogenase, 3-hydroxybutyryl-CoA dehydratase (crotonase), butyryl-CoA dehydrogenase, butyraldehyde dehydrogenase, and butanol dehydrogenase.

11. The microorganism of claim 10, wherein the butanol biosynthetic pathway gene is from a solventogenic bacteria.

12. The microorganism of claim 11, wherein the solventogenic bacteria is Clostridium acetobutylicum.

13. The microorganism of claim 10, wherein the microorganism comprises heterologous butanol biosynthetic pathway genes that encode acetyl-CoA C-acetyltransferase (thiolase), 3-hydroxybutyryl-CoA dehydrogenase, 3-hydroxybutyryl-CoA dehydratase (crotonase), butyryl-CoA dehydrogenase, butyraldehyde dehydrogenase, and butanol dehydrogenase.

14. The microorganism of claim 13, wherein the butanol biosynthetic pathway gene is from a solventogenic bacteria.

15. The microorganism of claim 14, wherein the solventogenic bacteria is Clostridium acetobutylicum.

16. The microorganism of claim 1, wherein a competing product pathway has been disrupted.

17. The microorganism of claim 16, wherein the competing product pathway is an ethanol pathway.

18. The microorganism of claim 17, wherein the ethanol pathway is disrupted by inactivating one or more alcohol dehydrogenases.

19. A method for the production of butanol from cellulose, comprising:
(a) providing a recombinant microorganism according to claim 1; and
(b) contacting the microorganism with cellulose under conditions whereby butanol is produced.

20. The method of claim 19, further comprising the step of isolating the butanol that is produced.

21. A recombinant microorganism, comprising:
(1) at least one heterologous butanol biosynthetic pathway gene that encodes a polypeptide that catalyzes a substrate to product conversion selected from the group consisting of:
(a) acetyl-CoA to acetoacetyl-CoA
(b) acetoacetyl-CoA to (S)-3-hydroxbutanoyl-CoA
(c) (S)-3-hydroxbutanoyl-CoA to crotonoyl-CoA
(d) crotonoyl-CoA to butyryl-CoA
(e) butyryl-CoA to butanal
(f) butanal to butanol;
(2) at least one heterologous gene that encodes a cellulase enzyme; and
(3) a heterologous gene that encodes a laccase polypeptide;
wherein said recombinant microorganism converts lignocellulose to butanol.

22. The microorganism of claim 21, wherein the gene that encodes the laccase polypeptide is the PDXA1b gene from Pleurotus ostreatus.

23. A method for the production of butanol from lignocellulose, comprising:
(a) providing a recombinant microorganism according to claim 21; and
(b) contacting the microorganism with lignocellulose under conditions whereby butanol is produced.

24. The method of claim 23, further comprising the step of isolating the butanol that is produced.

25.-33. (canceled)


SEQUENCE LISTING (13 sequences)

SEQ ID NO: 1 - DNA, 3368 bp, Artificial Sequence
  Description of Artificial Sequence Synthetic construct
aagcttgcatgcagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggatctgcaggtcgacatgcaactgttcaatttgccattgaaagtttcattctttctcgtcctctcttacttttctttgctcgtttctgctgactacaaggacgatgacgacaaatctagacaggcttgctcaagcgtctggggccaatgtggtggccagaattggtcgggtccgacttgctgtgcttccggaagcacatgcgtctactccaacgactattactcccagtgtcttcccggcgctgcaagctcaagctcgtccacgcgcgccgcatcgacgacttcacgagtatcccccacaacatcccggtcgagttccgcgacgcctccacctggttctactactaccagagtacctccagtcggatcgggaaccgctacgtattcaggcaacccttttgttggggtcactccttgggccaatgcatattacgcctctgaagttagcagcctcgctattcctagcttgactggagccatggccactgccgcagcagctgtcgcaaaggttccctcttttatgtggctagatactcttgacaagacccctctcatggagcaaaccttggccgacatccgcaccgccaacaagaatggcggtaactatgccggacagtttgtggtgtatgacttgccggatcgcgattgcgctgcccttgcctcgaatggcgaatactctattgccgatggtggcgtcgccaaatataagaactatatcgacaccattcgtcaaattgtcgtggaatattccgatatccggaccctcctggttattgagcctgactctcttgccaacctggtgaccaacctcggtactccaaagtgtgccaatgctcagtcagcctaccttgagtgcatcaactacgccgtcacacagctgaaccttccaaatgttgcgatgtatttggacgctggccatgcaggatggcttggctggccggcaaaccaagacccggccgctcagctatttgcaaatgtttacaagaatgcatcgtctccgagagcacttcgcggattggcaaccaatgtcgccaactacaacgggtggaacattaccagccccccatcgtacacgcaaggcaacgctgtctacaacgagaagctgtacatccacgctattggacgtcttcttgccaatcacggctggtccaacgccttcttcatcactgatcaaggtcgatcgggaaagcagcctaccggacagcaacagtggggagactggtgcaatgtgatcggcaccggatttggtattcgcccatccgcaaacactggggactcgttgctggattcgtttgtctgggtcaagccaggcggcgagtgtgacggcaccagcgacagcagtgcgccacgatttgactcccactgtgcgctcccagatgccttgcaaccggcgcctcaagctggtgcttggttccaagcctactttgtgcagcttctcacaaacgcaaacccatcgttcctgggatccagcgccaaaagctcttttatctcaaccactactactgatttaacaagtataaacactagtgcgtattccactggttccatttccacagtagaaacaggcaatcgaactacatcagaagtgatcagtcatgtggtgactaccagcacaaaactgtctccaactgctactaccagcctgacaattgcacaaaccagtatctattctactgactcaaatatcacagtaggaacagatattcacaccacatcagaagtgattagtgatgtggaaaccattagcagagaaacagcttcgaccgttgtagccgctccaacctcaacaactggatggacaggcgctatgaatacttacatcccgcaatttacatcctcttctttcgcaacaatcaacagcacaccaataatctcttcatcagcagtatttgaaacctcagatgcttcaattgtcaatgtgcacactgaaaatatcacgaatactgctgctgttccatctgaagagcccacttttgtaaatgccacgagaaactccttaaattccttttgcagcagcaaacagccatccagtccctcatcttatacgtcttccccactcgtatcgtccctctccgtaagcaaaacattactaagcaccagttttacgccttctgtgccaacatctaatacatatatcaaaacggaaaatacgggttactttgagcacacggctttgacaacatcttcagttggccttaattcttttagtgaaacagcactctcatctcagggaacgaaaattgacacctttttagtgtcatccttgatcgcatatccttcttctgcatcaggaagccaattgtccggtatccaacagaatttcacatcaacttctctcatgatttcaacctatgaaggtaaagcgtctatatttttctcagctgaactcggttcgatcatttttctgcttttgtcgtacctgctattctaacccgggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgagctcgaattc

SEQ ID NO: 2 - DNA, 2544 bp, Artificial Sequence
  Description of Artificial Sequence Synthetic construct
tctagagatgaactggcgttctctcctcctttctacccctctccgtgggccaatggccagggagagtgggcggaagcctaccagcgtgcagtggccattgtatcccagatgactctggatgagaaggtcaacctgaccaccggaactggatgggagctggagaagtgcgtcggtcagactggtggtgtcccaagactgaacatcggtggcatgtgtcttcaggacagtcccttgggtattcgtgatagtgactacaattcggctttccctgctggtgtcaacgttgctgcgacatgggacaagaaccttgcttatctacgtggtcaggctatgggtcaagagttcagtgacaaaggaattgatgttcaattgggaccggccgcgggtcccctcggcaggagccctgatggaggtcgcaactgggaaggtttctctccagacccggctcttactggtgtgctctttgcggagacgattaagggtattcaagacgctggtgtcgtggcgacagccaagcattacattctcaatgagcaagagcatttccgccaggtcgcagaggctgcgggctacggattcaatatctccgacacgatcagctctaacgttgatgacaagaccattcatgaaatgtacctctggcccttcgcggatgccgttcgcgccggcgttggcgccatcatgtgttcctacaaccagatcaacaacagctacggttgccagaacagttacactctgaacaaacttctgaaggccgaactcggcttccagggctttgtgatgtctgactggggtgctcaccacagtggtgttggctctgctttggccggcttggatatgtcaatgcctggcgatatcaccttcgattctgccactagtttctggggaaccaacctgaccattgctgtgctcaacggaaccgtcccgcagtggcgcgttgacgacatggctgtccgtatcatggctgcctactacaaggttggccgcgaccgcctgtaccagccgcctaacttcagctcctggactcgcgatgaatacggcttcaagtatttctacccccaggaagggccctatgagaaggtcaatcactttgtcaatgtgcagcgcaaccacagcgaggttattcgcaagttgggagcagacagtactgttctactgaagaacaacaatgccctgccgctgaccggaaaggagcgcaaagttgcgatcctgggtgaagatgctggttccaactcgtacggtgccaatggctgctctgaccgtggctgtgacaacggtactcttgctatggcttggggtagcggcactgccgaatttccatatctcgtgacccctgagcaggctattcaagccgaggtgctcaagcataagggcagcgtctacgccatcacggacaactgggcgctgagccaggtggagaccctcgctaaacaagccagtgtctctcttgtatttgtcaactcggacgcgggagagggctatatctccgtggacggaaacgagggcgaccgcaacaacctcaccctctggaagaacggcgacaacctcatcaaggctgctgcaaacaactgcaacaacaccatcgttgtcatccactccgttggacctgttttggttgacgagtggtatgaccaccccaacgttactgccatcctctgggcgggcttgcctggccaggagtctggcaactccttggctgacgtgctctacggccgcgtcaacccaggcgccaaatctccattcacctggggcaagacgagggaggcgtacggggattaccttgtccgtgaactcaacaacggcaacggagcaccccaagatgatttctcggaaggtgttttcattgactaccgcggattcgacaagcgcaatgagaccccgatctacgagttcggacatggtctgagctacaccactttcaactactctggccttcacatccaggttctcaacgcttcctccaacgctcaagtagccactgagactggcgccgctcccaccttcggacaagtcggcaatgcctctgactacgtgtaccctgagggattgaccagaatcagcaagttcatctatccctggcttaattccacagacctgaaggcctcatctggcgacccgtactatggagtcgacaccgcggagcacgtgcccgagggtgctactgatggctctccgcagcccgttctgcctgccggtggtggctctggtggtaacccgcgcctctacgatgagttgatccgtgtttcggtgacagtcaagaacactggtcgtgttgccggtgatgctgtgcctcaattgtatgtttcccttggtggacccaatgagcccaaggttgtgttgcgcaaattcgaccgcctcaccctcaagccctccgaggagacggtgtggacgactaccctgacccgccgcgatctgtctaactgggacgttgcggctcaggactgggtcatcacttcttacccgaagaaggtccatgttggtagctcttcgcgtcagctgccccttcacgcggcgctcccgaaggtgcaaggatcctaaggtacc

SEQ ID NO: 3 - DNA, 1212 bp, Artificial Sequence
  Description of Artificial Sequence Synthetic construct
tctagacagcagactgtctggggccagtgtggaggtattggttggagcggacctacgaattgtgctcctggctcagcttgttcgaccctcaatccttattatgcgcaatgtattccgggagccactactatcaccacttcgacccggccaccatccggtccaaccaccaccaccagggctacctcaacaagctcatcaactccacccactagctctggggtccgatttgccggcgttaacatcgcgggttttgactttggctgtaccacagatggcacttgcgttacctcgaaggtttatcctccgttgaagaacttcaccggctcaaacaactaccccgatggcatcggccagatgcagcacttcgtcaacgaggacgggatgactattttccgcttacctgtcggatggcagtacctcgtcaacaacaatttgggcggcaatcttgattccacgagcatttccaagtatgatcagcttgttcaggggtgcctgtctctgggcgcatactgcatcgttgacatccacaattatgctcgatggaacggtgggatcattggtcagggcggccctactaatgctcaattcacgagcctttggtcgcagttggcatcaaagtacgcatctcagtcgagggtgtggttcggcatcatgaatgagccccacgacgtgaacatcaacacctgggctgccacggtccaagaggttgtaaccgcaatccgcaacgctggtgctacgtcgcaattcatctctttgcctggaaatgattggcaatctgctggggctttcatatccgatggcagtgcagccgccctgtctcaagtcacgaacccggatgggtcaacaacgaatctgatttttgacgtgcacaaatacttggactcagacaactccggtactcacgccgaatgtactacaaataacattgacggcgccttttctccgcttgccacttggctccgacagaacaatcgccaggctatcctgacagaaaccggtggtggcaacgttcagtcctgcatacaagacatgtgccagcaaatccaatatctcaaccagaactcagatgtctatcttggctatgttggttggggtgccggatcatttgatagcacgtatgtcctgacggaaacaccgactggcagtggtaactcatggacggacacatccttggtcagctcgtgtctcgcaagaaagggatcctaaggtacc

SEQ ID NO: 4 - DNA, 69 bp, Artificial Sequence
  Description of Artificial Sequence Synthetic primer
gcacaatatttcaagctataccaagcatacaatcaactatctcatatacacagctgaagcttcgtacgc

SEQ ID NO: 5 - DNA, 72 bp, Artificial Sequence
  Description of Artificial Sequence Synthetic primer
ttttttataacttatttaataataaaaatcataaatcataagaaattcgcgcataggccactagtggatctg

SEQ ID NO: 6 - DNA, 69 bp, Artificial Sequence
  Description of Artificial Sequence Synthetic primer
aagatacctaagaaaattatttaactacatatctacaaaatcaaagcatccagctgaagcttcgtacgc

SEQ ID NO: 7 - DNA, 72 bp, Artificial Sequence
  Description of Artificial Sequence Synthetic primer
atagcttatataaaaagtaaaaatatattcatcaaattcgttacaaaagagcataggccactagtggatctg

SEQ ID NO: 8 - DNA, 20 bp, Artificial Sequence
  Description of Artificial Sequence Synthetic primer
tctctctcccccgttgttgt

SEQ ID NO: 9 - DNA, 20 bp, Artificial Sequence
  Description of Artificial Sequence Synthetic primer
ctcaggtaaggggctagtag

SEQ ID NO: 10 - DNA, 20 bp, Artificial Sequence
  Description of Artificial Sequence Synthetic primer
gcgccattcaagtcccgcga

SEQ ID NO: 11 - DNA, 21 bp, Artificial Sequence
  Description of Artificial Sequence Synthetic primer
caatttaaccaatttctactc

SEQ ID NO: 12 - DNA, 18 bp, Artificial Sequence
  Description of Artificial Sequence Synthetic primer
ggatgtatgggctaaatg

SEQ ID NO: 13 - DNA, 18 bp, Artificial Sequence
  Description of Artificial Sequence Synthetic primer
cctcgacatcatctgccc